(19) United States
(12) Patent Application Publication
(10) Pub. No.: US 2023/0414479 A1
(43) Pub. Date: Dec. 28, 2023

(54) DETOX HAIR AND SCALP CLEANSER COMPOSITION, METHODS AND USES THEREOF

(71) Applicant: K18, Inc., San Francisco, CA (US)

(72) Inventors: Artur Manuel CAVACO PAULO, Braga (PT); Karis STALEY, Huntington Beach, CA (US); Meagan SCHLAPP, Maspeth, NY (US)

(21) Appl. No.: 18/339,927

(22) Filed: Jun. 22, 2023

Related U.S. Application Data
(60) Provisional application No. 63/354,516, filed on Jun. 22, 2022.

(51) Int. Cl. A61K 8/64; A61K 8/19; A61Q 5/12; A61Q 5/02

(52) U.S. Cl. CPC A61K 8/64; A61K 8/19; A61Q 5/12; A61Q 5/02; A61K 2800/51; A61K 2800/524; A61K 2800/596

(57) ABSTRACT

The present disclosure provides a detoxifying hair and scalp cleanser composition for cleansing and repairing hair. The detoxifying hair and scalp cleanser compositions described herein have a pH optimized to cleanse the hair and scalp. The pH of the detoxifying hair and scalp cleanser composition minimizes reduces swelling and protein loss. The detoxifying hair and scalp cleanser compositions described herein comprise peptides, which are based on the structure of keratin and keratin associated proteins. The peptides described bind hair with high affinity and repair keratinous fiber damage through the formation of disulfide bonds and other bonding interactions such as hydrogen bonds, ionic bonds, and Van der Waals interactions.

### CROSS-REFERENCE

This application claims the benefit of U.S. Provisional Application No. 63/354,516, filed Jun. 22, 2022, which is hereby incorporated by reference in its entirety herein.

### BACKGROUND

Hair and scalp cleansing is a common grooming practice considered important for optimal hair cleanliness, health, and maintenance. Hair cleansers typically remove sebum, sweat, desquamated stratum corneum, and environmental dirt, which helps prevent development of conditions such as folliculitis and seborrheic dermatitis. Current commercial formulas employ synthetic detergents, technically described as surfactant systems, blended in varying quantities and ratios to achieve the desired amount of cleansing power. The cosmetic industry has provided consumers innumerable options that facilitate general hair cleansing, encompassing formulas with a broad variety of ingredients and compositions.

In addition to removing sebum, sweat, desquamated stratum corneum, and environmental dirt, a secondary function of shampoos is to impart a desired sensory effect to the hair. Hair that has been thoroughly cleaned—i.e., is completely devoid of any sebum or products—tends to feel less smooth and look duller and as a result is generally not considered cosmetically desirable. One strategy to counter this problem is to incorporate a multitude of conditioners into the cleanser. These conditioners are deposited onto the hair surface, and generally modify surface properties of the hair only—improving the look and feel of hair but do not address any underlying damage to the structure of hair. The excess deposition of these conditioning agents, especially when combined with inadequate cleansing, can cause the conditioners to accumulate in the hair and weigh hair down or make it appear duller. Commercial products typically focus on providing benefit to either the hair or the scalp. However, hair's health begins at the follicle and the treatments and products applied to hair impact the scalp; the two are inextricably linked. Products addressing the holistic health of the hair and scalp system will provide the most benefits. Thus, there is a need for new hair cleanser compositions which both cleans the hair and scalp provides desired hair properties without excess deposition of conditioning agents.

### SUMMARY

In one aspect, the present disclosure provides a detoxifying hair and scalp cleanser composition for cleaning and repairing hair. In some embodiments, the detoxifying hair and scalp cleanser composition comprises one or more solvents, one or more peptides, one or more hair conditioners, one or more skin conditioners, one or more detoxifying agents, one or more cleansing agents; and one or more cosmetically or dermatologically acceptable excipients. In some embodiments, the one or more peptides comprises a peptide sequence comprising SEQ ID NO: 75, SEQ ID NO: 400, SEQ ID NO: 401, SEQ ID NO: 402, SEQ ID NO: 409, SEQ ID NO: 412, SEQ ID NO: 432, SEQ ID NO:1131, or a combination thereof. In some embodiments, the one or more peptides comprises a peptide sequence comprising SEQ ID NO: 400, SEQ ID NO: 401, SEQ ID NO: 409, SEQ ID NO: 412, SEQ ID NO: 432, or a combination thereof. In some embodiments, the one or more peptides comprises a peptide sequence comprising SEQ ID NO: 400, SEQ ID NO: 409, SEQ ID NO: 412, or a combination thereof. In some embodiments, the one or more peptides comprises SEQ ID NO: 400. In some embodiments, the one or more peptides comprises SEQ ID NO: 409. In some embodiments, the one or more peptides comprises SEQ ID NO: 412.

In some embodiments, the concentration (% w/w) of the one or more peptides in the detoxifying hair and scalp cleanser is between about 0.0001% to about 1%. In some embodiments, the concentration (% w/w) of the one or more peptides in the detoxifying hair and scalp cleanser is between about 0.0001% to about 0.01%. In some embodiments, the concentration (% w/w) of the one or more peptides in the detoxifying hair and scalp cleanser is between about 0.0001% to about 0.001%.

In some embodiments, the detoxifying hair and scalp cleanser composition includes one or more hair conditioners selected from: panthenol, salicylic acid, Rubus ideaus (raspberry) leaf extract, Carthamus tinctorius (safflower) seed extract, hydroxypropylgluconamide, hydroxypropylammonium gluconate, hydrolyzed hyaluronic acid, sodium hyaluronate, or combinations thereof. In some embodiments, the detoxifying hair and scalp cleanser composition includes one or more hair conditioners selected from: panthenol and salicylic acid. In some embodiments, the one or more hair conditioners comprises panthenol. In some embodiments, the one or more hair conditioners comprises salicylic acid.

In some embodiments, the detoxifying hair and scalp cleanser composition includes one or more skin conditioners selected from: caprylyl glycol, maltooligosyl glucoside, hydrogenated starch hydrolysate, Opuntia ficus-indica stem extract, saccharide isomerase, alpha-glucan oligosaccharide, Alteromonas ferment extract, Anetholea anisata leaf extract, guar hydroxypropyltriammonium chloride, or combinations thereof. In some embodiments, the detoxifying hair and scalp cleanser composition includes one or more skin conditioners selected from: caprylyl glycol and guar hydroxypropyltriammonium chloride. In some embodiments, the one or more skin conditioners comprises caprylyl glycol. In some embodiments, the one or more skin conditioners comprises guar hydroxypropyltriammonium chloride.

In some embodiments, the detoxifying hair and scalp cleanser composition includes one or more detoxifying agents. In some embodiments, the one or more detoxifying agents is an absorbent. In some embodiments, the absorbent is a charcoal powder.

In some embodiments, the detoxifying hair and scalp cleanser composition includes one or more cleansing agents. In some embodiments, the one or more cleansing agents is a surfactant. In some embodiments, the surfactant is selected from: an anionic surfactant, an amphoteric surfactant, a non-ionic surfactant, or combinations thereof.

In some embodiments, the surfactant includes at least one anionic surfactant selected from: sodium methyl 2-sulfolaurate, sodium lauryl sulfoacetate, disodium 2-sulfolaurate, alkylbenzene sulfonates, ammonium lauryl sulfate, ammonium lauryl sulfate, ammonium xylenesulfonate, sodium C₁₄₋₁₆ olefin sulfonate, sodium cocoyl sarcosinate, sodium laureth sulfate, sodium lauryl sulfate, sodium lauryl sulfoacetate, sodium myreth sulfate, sodium xylenesulfonate, TEA-dodecylbenzenesulfonate, ethyl PEG-15 cocamine sulfate, dioctyl sodium sulfosuccinate, or combinations thereof. In some embodiments, the at least one anionic surfactant comprises sodium C₁₄₋₁₆ olefin sulfonate.

In some embodiments, the surfactant includes at least one amphoteric surfactant selected from: cocamindopropyl hydroxysultaine, cocamidopropyl betaine, coco betaine, cocoamphoacetate, cocoamphodipropionate, disodium cocoamphodiacetate, disodium cocoamphodipropionate, lauroamphoacetate, sodium cocoyl isethionate, or combinations thereof. In some embodiments, the at least one anionic surfactant comprises cocamindopropyl hydroxysultaine.

In some embodiments, the detoxifying hair and scalp cleanser composition includes one or more cosmetically or dermatologically acceptable excipients selected from: a humectant; a chelating agent; a buffer or a preservative, or combinations thereof.

In some embodiments, the humectant is selected from: glycerin, propylene glycol, propanediol, or isopentyldiol. In some embodiments, the humectant comprises glycerin.

In some embodiments, the chelating agent is selected from: sodium phytate, EDTA, sodium gluconate, trisodium dicarboxymethyl alaninate, tetrasodium glutamate diacetate, or caprylhydroxamic acid, or combinations thereof. In some embodiments, the chelating agent comprises caprylhydroxamic acid, sodium phytate, or combinations thereof. In some embodiments, the chelating agent comprises caprylhydroxamic acid. In some embodiments, the chelating agent comprises sodium phytate.

In some embodiments, the buffer is selected from: citric acid, lactic acid, glycolic acid, malic acid, tartaric acid, L-arginine, sodium hydroxide, potassium hydroxide, or combinations thereof. In some embodiments, the buffer comprises citric acid, potassium hydroxide, or combinations thereof. In some embodiments, the buffer comprises citric acid. In some embodiments, the buffer comprises potassium hydroxide.

In some embodiments, the preservative is selected from: phenoxyethanol, caprylhydroxamic acid, potassium sorbate, ethylhexylglycerin, gluconolactone, sodium benzoate, benzoic acid, or combinations thereof.

In some embodiments, the concentration (% w/w) of the one or more solvents or in the detoxifying hair and scalp cleanser composition is between about 50% to about 90%. In some embodiments, the concentration (% w/w) of the one or more hair conditioners in the detoxifying hair and scalp cleanser composition is between about 0.01% to about 10%. In some embodiments, the concentration (% w/w) of the one or more skin conditioners in the detoxifying hair and scalp cleanser composition is between about 0.01% to about 5%. In some embodiments, the concentration (% w/w) of the one or more cleansing agents in the detoxifying hair and scalp cleanser composition is between about 0.1% to about 20%. In some embodiments, the concentration (% w/w) of the one or more cosmetically or dermatologically acceptable excipients in the detoxifying hair and scalp cleanser composition is between about 0.1% to about 30%. In some embodiments the one or more solvents comprises water.

In some embodiments, the pH of the detoxifying hair and scalp cleanser composition is between about 4 and about 6. In some embodiments, the pH of the detoxifying hair and scalp cleanser composition is about 4. In some embodiments, the pH of the detoxifying hair and scalp cleanser composition is about 4.5. In some embodiments, the pH of the detoxifying hair and scalp cleanser composition is about 5. In some embodiments, the pH of the detoxifying hair and scalp cleanser composition is about 5.5. In some embodiments, the pH of the detoxifying hair and scalp cleanser composition is about 6.

In another aspect, a method for cleansing hair using the detoxifying hair and scalp cleanser composition described herein is provided.

Additional aspects and advantages of the present disclosure may become readily apparent to those skilled in this art from the following detailed description, wherein only illustrative embodiments of the present disclosure are shown and described. As may be realized, the present disclosure is capable of other and different embodiments, and its several details are capable of modifications in various obvious respects, all without departing from the disclosure.

### DETAILED DESCRIPTION

Human hair fiber structure consists of cuticle, cortex and medulla. The cuticle consists of a thin overlapping layer of cells, like a system of scales, and provides protection for the inner core of the hair, known as the cortex. Cuticle cells are mechanically robust and are resistant to many environmental factors, including the penetration of foreign molecules, due to the high degree of isodipeptide and disulfide bonds present in its various lamellar layers. The cuticle represents roughly 15% of the weight of a strand of hair. The cortex is the middle region of the hair and represents about 80% of the weight of a strand of hair. The cortex is composed of hierarchal structures of elongated, fibrous alpha-helical keratin extending along the fiber axis embedded in an amorphous protein matrix. It is the cortex with its system of intertwined protein fibers and supporting matrix that gives hair its tensile properties (strength, elasticity) of hair. A cell membrane complex joins cuticle cells with other cuticle cells, cortical cells with other cortical cells, and cuticle cells with cortical cells. The medulla corresponds to a central beam of cells and is absent in some hair fibers.

Human hair comprises approximately 65% to 95% protein. Proteins exhibit their active properties within specific temperature and pH value ranges and can unfold beyond these limits. Thermal denaturation for fibrous hard alpha-keratin proteins, i.e., the cortical proteins responsible for hair strength and elasticity, show a significant increase in stability when treated with low pH solution. This is particularly true for those with oxidative damage, such as by bleaching, and has been evidenced by a significant increase in enthalpy and peak temperature of thermal denaturation as quantified by DSC. Enthalpy is generally understood to reflect the quantity and structural integrity of alpha-helical material in hair intermediate filaments (IFs), while the peak temperature reflects the cross-link density of the matrix in which IFs are embedded. This phenomenon is further supported by amino acid analysis, X-ray diffraction, Raman spectroscopy, and tensile strength observations.

Alkaline pH creates a more favorable environment for cystine (disulfide) disruption as well as hydrolysis of amide bonds and formation of lanthionine and lysinoalanine—all of which weaken the hair's protein structure. Duration of the exposure of hair to solutions of a particular pH does not significantly change thermal stability; when compounded by the fact that tap water pH varies broadly from neutral to alkaline, this suggests that negative effects may be experienced in the duration of time for which hair is reasonably expected to be exposed during normal washing conditions.

The observed differences in thermal stability of hair proteins exposed to varied pH is due to a change in environment of the intermediate filaments, i.e., the interface between IFs and surrounding matrix proteins. This scaffolding structure at the IF surface is made by side chain interactions that anchor filaments to matrix. Strong adsorption of protons at the interface of the IFs may help shield this interface; disulfide bonds as well as electrostatic forces further stabilize this region.

During cleansing, the hair is in a structurally vulnerable state because water molecules break the hydrogen bonds that stabilize the protein structure. This causes swelling of certain parts of the cuticle and predisposes the hair fiber to damage. Further, it is established that the swelling behavior of the hair cuticle is highly dependent on pH: the more alkaline the environment, the greater the degree of swelling. Aside from the impact on thermal stability, this modification of cuticular swelling by pH has implications toward hair's properties and propensity for damage that is directly relevant for consumers. When the cuticle is swelled hair fibers have increased inter-fiber frictional force, resulting in increased tangling and a rough, unpleasant feel. Hair is also in a weaker state when wet; the need for greater comb force combined with the lower break stress of wet-state hair can easily lead to cuticle chipping, protein loss, and even critical breakage during hair washing and shampooing. Minimization of cuticle swelling exhibited at acidic pH may help offset this effect.

Proteins in hair are in an optimal state (most structurally resilient) around a pH of 5. This more acidic pH environment also minimizes excess swelling of the cuticle, reducing protein loss, preserving color, and minimizing electrostatic repulsion. This is also the ideal pH range for skin, which has an acidic mantle. Skin that has a disrupted pH can experience barrier perturbation, inflammation, and irritation. The pH of tap waters varies significantly from 6.5-9.53 and can contain metal ions like divalent calcium and magnesium (hard water minerals) which impact hair texture as well as trace levels of transition metals like copper, iron, nickel, lead, and others that can contribute to oxidative degradation of the hair fiber.

Further, peptides may be used to treat damaged hair during cleansing. These peptides may interact with the hair primarily via covalent (disulfide) bonds, polar bonds, and hydrophobic interactions. However, penetration of the peptide into the cortex of the hair strands, which is necessary for successful treatment, can be difficult. Molecular size and polarity play a large role in determining the ability of a molecule to gain access to the interior of the fiber structure. Further, the presence of sebum and products on the hair stands impedes the ability of the peptide to migrate successfully to the cortex. While appropriately acidic pH aids in the stabilization of hair's protein structure as specified above, these peptides may work in a complementary and synergistic way to add additional reinforcement. Taken together, this system provides both protection and repair of hair's protein structure which can be vulnerable during the washing process.

To ensure no residue remains on hair fiber that can interfere with peptide's ability to penetrate into the cortex, optimizing protein structural reinforcement and strengthening benefits, a clarifying shampoo should be used prior to application of reparative treatments comprising the peptide. Mineral buildup may also interfere with the peptide's efficacy, so the formula must be potent enough to remove 1) product, 2) sebum, 3) hard water mineral, and any other kinds of buildup from the hair. Salicylic acid can be challenging to formulate with in surfactant systems, so selection of potent, acid-compatible surfactants that are also ion-tolerant and stable in hard water aids in achievement of the three above types of buildup. Detoxifying agents such as, for example, charcoal sequester metal ions at low levels without further complicating formulation with viscosity-altering chelating agents. Charcoal contains many small pores that create a large surface area capable of adsorbing heavy metals including iron (Fe), chromium (Cr), cadmium (Cd), and copper (Cu).

Accordingly, provided herein is a detoxifying hair and scalp cleanser composition having an optimized pH that reduces swelling and protein loss. Formulating a shampoo at more acidic pH can help counteract this alkaline pH environment and minimize the net negative charge on hair proteins, which minimizes cuticle swelling and also reduces binding sites for dissolved metal cations. While having protective agents like emollients, occlusives, cationics, and polymers on the hair fiber minimizes friction and associated styling damage and provides a barrier to heat, UV, and pollution, excessive product build-up which can weigh hair down and prevent additional products from performing as well. The hair compositions may include peptides which reinforce the protein structure within the hair cortex to restore tensile strength to hair fibers. These peptides may not be compatible with traditional conditioning agents that form a film or coating and can block the peptide from interacting with hair proteins and most especially from entry to the cortex.

In some embodiments, the detoxifying hair and scalp cleanser compositions described herein comprise low molecular weight peptides capable of penetrating to the cortex of the hair and improving the mechanical properties of hair. In some embodiments, the detoxifying hair and scalp cleanser compositions described herein provide a long-lasting improved texture and improved manageability to the hair when dried. After prolonged use, the detoxifying hair and scalp cleanser compositions described herein provide the hair with soft, shiny, undamaged texture and with the other desired features. The detoxifying hair and scalp cleanser compositions described herein do not strip the hair of any essential oils, mitigates protein loss, and repairs the underlying hair damage, thus avoiding excess reliance on conditioners that coat the hair fiber to achieve desired properties. Further, due to their limited ingredients, the detoxifying hair and scalp cleanser compositions described herein do not contribute to the build-up of excess conditioning agents or other products that can ultimately weigh hair down.

In some embodiment, the detoxifying hair and scalp cleanser composition provided herein comprise one or more solvents, one or more peptides, one or more hair conditioners, one or more skin conditioners, one or more cleansing agents, and one or more cosmetically or dermatologically acceptable excipients. In some embodiments, the one or more peptides have a peptide sequence from 6 to 15 amino acids. In some embodiments, from 2 to 5 amino acids of the peptide sequence are cysteines. The composition of the peptides provides improved resistance, strength, toughness, and elasticity to the hair.

In striving to employ a minimalist approach of “less-is-more,” selection of multifunctional ingredients is key. Salicylic acid has a well-established efficacy in skincare, providing desmolytic and comedolytic activity to promote healthy cell turnover as well as balancing sebaceous production, conferring photoprotection, and providing anti-microbial and anti-inflammatory effects. While these properties are expected to translate to scalp, the compound exhibits further scalp-specific benefits: it is useful when applied topically in the treatment of conditions such as dandruff, ichthyosis, and psoriasis.³¹ Its pKa of approximately 2.8 and associated low pH make salicylic acid an ideal ingredient for application to the hair fiber in addition to the acidic pH benefits described above.

The detoxifying hair and scalp cleanser composition described herein cleans the hair while maintaining an optimum pH environment of hair to minimize fiber swelling and preserve structural integrity. Additionally, the hair cleanser compositions clean the scalp while maintaining an optimum pH environment to avoid over drying the scalp and without disrupting the natural barrier function of the scalp. The detoxifying hair and scalp cleanser compositions described herein have a pH optimized to cleanse the hair and scalp. In some embodiments, the pH of the detoxifying hair and scalp cleanser composition is optimized to promote the efficacy of certain ingredients in the compositions. In some embodiments, the pH of the detoxifying hair and scalp cleanser composition is optimized to promote the efficacy of salicylic acid. In some embodiments, the pH of the composition is optimized to promote chelation of certain minerals and metals. In some embodiments, the pH of the composition is optimized to minimize swelling of the cuticle. In some embodiments, reduced swelling of the cuticle reduces tangling of the hair. In some embodiments, the pH of the composition is optimized to protect the structural integrity of the hair and mitigate protein and lipid loss.

In some embodiments, the detoxifying hair and scalp cleanser composition has a pH between about 1 to about 7. In some embodiments, the detoxifying hair and scalp cleanser composition has a pH of about 1, about 1.25 about 1.5, about 1.75, about 2, about 2.25, about 2.5, about 2.75, about 3, about 3.25, about 3.5, about 3.75, about 4, about 4.25, about 4.5, about 4.75, about 5, about 5.25, about 5.5, about 5.75, about 6, about 6.25, about 6.5, about 6.75, about 7, about, and any values therebetween. In some embodiments, the detoxifying hair and scalp cleanser composition has a pH between about 1 to about 7, between about 2 to about 7, between about 3 to about 7, between about 3 to about 6, between about 4 to about 6, and any values therebetween.

### Definitions

Unless defined otherwise, all terms of art, notations, and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range of formats. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, a description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including combinations thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative, or quantitative and qualitative determinations. Assessing can be relative or absolute. “Detecting the presence of” can include determining the amount of something present in addition to determining whether it is present or absent depending on the context.

The term “about” or “approximately” as used herein when referring to a measurable value such as an amount or concentration and the like, is meant to encompass variations of 20%, 10%, 5%, 1%, 0.5%, or even 0.1% of the specified amount. For example, “about” can mean plus or minus 10%, per the practice in the art. Alternatively, “about” can mean a range of plus or minus 20%, plus or minus 10%, plus or minus 5%, or plus or minus 1% of a given value. Alternatively, particularly with respect to biological systems or processes, the term can mean within an order of magnitude, up to 5-fold, or up to 2-fold, of a value. Where particular values can be described in the application and claims, unless otherwise stated the term “about” may be assumed to encompass the acceptable error range for the particular value. Also, where ranges, subranges, or both, of values can be provided, the ranges or subranges can include the endpoints of the ranges or subranges.

Where values are described as ranges, it may be understood that such disclosure includes the disclosure of all possible sub-ranges within such ranges, as well as specific numerical values that fall within such ranges irrespective of whether a specific numerical value or specific sub-range is expressly stated.

The terms “comprise,” “have,” and “include” are open-ended linking verbs. Any forms or tenses of one or more of these verbs, such as “comprises,” “comprising,” “has,” “having,” “includes,” and “including,” are also open-ended. For example, any method that “comprises,” “has,” or “includes” one or more steps is not limited to possessing only those one or more steps and also covers other unlisted steps.

As used herein, the term “mixture” or “mixtures” is meant to include a simple combination of materials and any compounds that may result from their combination.

As used herein, the term “cosmetically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

As used herein, the term “cosmetically or dermatologically acceptable” refers to the compositions or components described are suitable for use in contact with human skin tissue without undue toxicity, incompatibility, instability, allergic response, and the like.

The section headings used herein are for organizational purposes only and are not to be construed as limiting the subject matter described.

### Peptides

Keratin proteins may be lost when washed with a standard shampoo. Further, the pH of the hair during the washing may further contribute to protein loss due to swelling of the hair cuticle. The present application describes a detoxifying hair and scalp cleanser composition that comprises peptides, which are based on the structure of keratin and keratin associated proteins. The peptides described herein bind hair with high affinity and repair keratinous fiber damage through the formation of disulfide bonds. The peptides have a sequence containing one or more cysteine amino acids that form disulfide bonds with free cysteine amino acids in the hair or keratins as well as participate in other types of bonding interactions that stabilize native hair proteins. The formation of new peptide-protein bonds helps to restore the mechanical properties of the hair fiber including elasticity, resistance, and appearance.

In some embodiments, the peptides described herein have a low molecular weight, between 6 to 15 amino acids, improving their ability to diffuse into the hair cortex to reduce protein damage and loss during repeated washing and chemical treatments and improve the physical and mechanical properties of the hair fibers.

In some embodiments, the peptides described herein include hydrophobic residues for added hydrophobicity and improved affinity to hair proteins. In some embodiments, the peptides described herein include polar residues for added hydrophilicity and improved affinity to hair proteins.

In some embodiments, each of the peptide sequences described herein are used separately or in combination, as well as all or part of the peptide sequence. In some embodiments, the detoxifying hair and scalp cleanser composition comprises at least one or more peptide sequences. In some embodiments, the detoxifying hair and scalp cleanser composition comprises at least two or more peptide sequences. In some embodiments, the detoxifying hair and scalp cleanser composition comprises at least three or more peptide sequences. In other embodiments, the detoxifying hair and scalp cleanser composition comprises at least four or more peptide sequences.

The number of amino acids of the peptide sequence is from 6 to 15. In some embodiments, the number of amino acids of the peptide sequence is 6. In some embodiments, the number of amino acids of the peptide sequence is 7. In some embodiments, the number of amino acids of the peptide sequence is 8. In some embodiments, the number of amino acids of the peptide sequence is 9. In some embodiments, the number of amino acids of the peptide sequence is 10. In some embodiments, the number of amino acids of the peptide sequence is 11. In some embodiments, the number of amino acids of the peptide sequence is 12. In some embodiments, the number of amino acids of the peptide sequence is 13. In some embodiments, the number of amino acids of the peptide sequence is 14. In some embodiments, the number of amino acids of the peptide sequence is 15.

In some embodiments, each peptide sequence contains amino acids comprising sulfur. In some embodiments, the peptide sequence comprises cysteine amino acids, that interact with the hair and allows the formation of intermolecular cross-linking, stabilizing the keratinous fiber. In some embodiments, the detoxifying hair and scalp cleanser composition described herein comprises peptide sequences with high cysteine content to enrich the properties of the hair, such as to improve elasticity and resistance, reduce putative damage of the hair, improve and/or change hair characteristics. In some embodiments, cysteine comprises 10% to 50% of the total amount of amino acids of the peptide sequence. In some embodiments, the peptide sequence comprises 2 cysteines. In some embodiments, the peptide sequence comprises 3 cysteines. In some embodiments, the peptide sequence comprises 4 cysteines. In some embodiments, the peptide sequence comprises 5 cysteines.

In some embodiments, the peptides comprise a peptide sequence comprising SEQ ID NO: 75, SEQ ID NO: 400, SEQ ID NO: 401, SEQ ID NO: 402, SEQ ID NO: 409, SEQ ID NO: 412, SEQ ID NO: 432, SEQ ID NO: 951, SEQ ID NO:1131, or combinations thereof. In some embodiments, the peptides comprise a peptide sequence comprising at least 90%, 91%, 92%, 93%, 94%, 95%, 96%, 97%, 98%, 99%, or 100% of SEQ ID NO: 75, SEQ ID NO: 400, SEQ ID NO: 401, SEQ ID NO: 402, SEQ ID NO: 409, SEQ ID NO: 412, SEQ ID NO: 432, SEQ ID NO: 951, SEQ ID NO:1131, or combinations thereof.

TABLE 1
Sequences of peptides

| SEQ ID NO | Sequences |
|---|---|
| 75 | CLPCL PAASC |
| 400 | GGV CGPSP PCITT |
| 401 | F CGFPS CSTSC |
| 402 | CT PPSCC QLHHA |
| 409 | GGVCG PSPPC |
| 412 | CGPSP PCITT |
| 432 | CEPAI CEPSC |
| 951 | SCCAP VYCCK |
| 1131 | C QCSCC KPYCS |

The concentration of the peptide to be used depends on several factors, such as the condition of the hair, the origin of the hair, and the hair treatment formulation. The peptides described herein can be prepared by conventional methods of peptide synthesis, well known in the state of the art. In some embodiments, the concentration (% w/w) of the one or more peptides in the detoxifying hair and scalp cleanser composition is between about 0.0001% to about 1.0%. In some embodiments, the concentration of the peptide in the detoxifying hair and scalp cleanser composition is about 0.0001%, about 0.0005%, about 0.0010%, about 0.0015%, about 0.0020%, about 0.0025%, about 0.0030%, about 0.0035%, about 0.0040%, about 0.0045%, about 0.0050%, about 0.0055%, about 0.0060%, about 0.0065%, about 0.0070%, about 0.0075%, about 0.0080%, about 0.0085%, about 0.0090%, about 0.0095%, about 0.01%, about 0.05%, about 0.055%, about 0.060%, about 0.065%, about 0.070%, about 0.075%, about 0.080%, about 0.085%, about 0.090%, about 0.095%, about 0.1%, about 0.15%, about 020%, about 0.25%, about 0.30%, about 0.35%, about 0.40%, about 0.45%, about 0.50%, about 0.55%, about 0.60%, about 0.65%, about 0.70%, about 0.75%, about 0.80%, about 0.85%, about 0.90%, about 0.95%, about 1.0%, and any values therebetween. In some embodiments, the concentration of the peptide in the detoxifying hair and scalp cleanser composition is between about 0.0001% to about 0.95%, between about 0.0005% to about 0.9%, between about 0.001% to about 0.85%, between about 0.005% to about 0.8%, between about 0.010% to about 0.75%, between about 0.050% to about 0.70%, between about 0.10% to about 0.65%, between about 0.15% to about 0.60%, between about 0.20% to about 0.55%, between about 0.25% to about 0.50%, between about 0.30% to about 0.45%, between about 0.35% to about 0.40%, and between about 0.40% to about 1.0%.

The detoxifying hair and scalp cleanser compositions described herein enrich and improve the properties and characteristics of the hair, such as elasticity, strength, pH, structural integrity, appearance, and feel, repairing and/or preserving damaged keratinous fiber. The detoxifying hair and scalp cleanser composition described herein increases resistance due to the presence of the cysteine-rich peptides of the composition, even after several rinsing's. The detoxifying hair and scalp cleanser compositions described herein acts as an effective cleanser without the associated harshness of other compositions in the art, at a hair-appropriate pH, and without excessive use of conditioning agents. In the context of the present disclosure, the detoxifying hair and scalp cleanser composition can also be applied to the hair as, but not limited to, an aqueous solution or conventional shampoo or conditioner.

### Hair Conditioners

The detoxifying hair and scalp cleanser composition described herein includes one or more hair conditioners which repair and improve the properties of hair, such as elasticity, resistance, texture (i.e., softness, smoothness, etc.), and appearance. In some embodiments, the detoxifying hair and scalp cleanser composition comprises one or more hair conditioners selected from: panthenol, salicylic acid, sodium salicylate, carnosine, tetrahexyldecyl ascorbate, hyaluronic acid/sodium hyaluronate, hydrolyzed hyaluronic acid, sarcosine, PCA, arginine PCA, brassicyl valinate esylate, brassicyl isoleucinate esylate, brassica alcohol, glycolipids, glycosphingolipids, sphinganine, Rubus ideaus (raspberry) leaf extract, bis-ethyl(isostearylimidazoline) isostearamide, Camellia sinensis leaf extract, Carthamus tinctorius (safflower) seed extract, Glycine soja (soybean) seed extract, Elaeis guineensis (palm) fruit extract, Oryza sativa (rice) extract, hydrolyzed Adansonia digitata seed extract, hydrolyzed corn starch, behenyl/stearyl aminopropanediol esters, C10-40 isoalkylamidopropylethyldimonium ethosulfate, Linum usitatissimum (linseed) extract, Salva hispanica seed extract, Astrocaryum mururmuru fruit extract, Moringa oleifera seed extract, cinnamidopropyltrimonium chloride, hydroxypropyl guar hydroxypropyltrimonium chloride, Trifoliumpratense (clover) flower extract, Opuntia ficus-indica stem extract, Kappaphycus alvarezii extract, Laminaria saccharina extract, guar hydroxypropyltriammonium chloride, bis-diglyceryl polyacyladipate-2, hydroxypropylgluconamide, hydroxypropylammonium gluconate, biosaccharide gum-4, prunus Amygdalus dulcic (sweet almond) seed extract, soybean glycerides, Butyrospermum parkii (shea) butter unsaponifiables, hydrolyzed chestnut extract, glyceryl rosinate, sodium methoxy peg-16 maleate/styrene sulfonate copolymer, panthenyl hydroxypropyl steardimonium chloride, and any combinations thereof.

In some embodiments, the concentration (% w/w) of the one or more hair conditioners in the detoxifying hair and scalp cleanser composition is between about 0.01% to about 10.0%. In some embodiments, the concentration of the one or more hair conditioners in the detoxifying hair and scalp cleanser is about 0.01%, about 0.05%, about 0.10%, about 0.15%, about 0.20%, about 0.25%, about 0.30%, about 0.35%, about 0.40%, about 0.45%, about 0.50%, about 0.55%, about 0.60%, about 0.65%, about 0.7%, about 0.75%, about 0.80%, about 0.85%, about 0.90%, about 0.95%, about 1.0%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, and any values therebetween. In some embodiments, the concentration of the one or more hair conditioners in the detoxifying hair and scalp cleanser composition is between about 0.01% to about 10%, between about 0.05% to about 9.5%, between about 0.20% to about 9%, between about 0.30% to about 8.5%, between about 0.40% to about 8%, between about 0.50% to about 7.5%, between about 0.60% to about 7%, between about 0.70% to about 6.5%, between about 0.80% to about 6%, between about 0.90% to about 5.5%, and between about 1% to about 5%.

### Skin Conditioners

The detoxifying hair and scalp cleanser composition described herein includes one or more skin conditioners which support the skin's barrier function and help retain moisture. In some embodiments, the detoxifying hair and scalp cleanser composition comprises one or more skin conditioners selected from: niacinamide, allantoin, maltooligosyl glucoside, hydrogenated starch hydrolysate, Opuntia ficus-indica stem extract, sodium hyaluronate crosspolymer, jojoba esters, hydrolyzed jojoba esters, oleic acid, linoleic acid, linolenic acid, lactobionic acid, ursolic acid/sodium ursolate, nordihydroguaiaretic acid, ceramides, fructooligosaccharides, galactoarabinan, schizophyllan, Anetholea anisate leaf extract, Tetraselmis suecica extract, saccharide isomerase, Alteromonas ferment extract, Pseudoalteromonas ferment extract, Corylus avellana leaf extract, Phyllanthus emblica fruit extract, Betula alba juice, Saccharum officinarum (sugarcane) extract, Rhodomyrtus tomentosa fruit extract, Hedychium coronarium root extract, Epilobium angustifolium flower/leaf/stem extract, Tremella fuciformis sporocarp extract, alpha-glucan oligosaccharide, and any combinations thereof.

In some embodiments, the concentration (% w/w) of the one or more skin conditioners in the detoxifying hair and scalp cleanser composition is between about 0.01% to about 5.0%. In some embodiments, the concentration of the one or more skin conditioners in the detoxifying hair and scalp cleanser composition is about 0.01%, about 0.05%, about 0.10%, about 0.15%, about 0.20%, about 0.25%, about 0.30%, about 0.35%, about 0.40%, about 0.45%, about 0.50%, about 0.55%, about 0.60%, about 0.65%, about 0.70%, about 0.75%, about 0.80%, about 0.85%, about 0.90%, about 0.95%, about 1.0%, and any values therebetween. In some embodiments, the concentration of the one or more skin conditioners in the detoxifying hair and scalp cleanser composition is between about 0.01% to about 0.95%, between about 0.05% to about 1.0%, between about 0.20% to about 0.90%, between about 0.30% to about 0.85%, between about 0.40% to about 0.80%, between about 0.50% to about 0.75%, between about 0.60% to about 0.70%, and between about 0.70% to about 1.0%.

### Detoxifying Agents

The detoxifying hair and scalp cleanser composition described herein includes one or more detoxing agents which may remove dirt, oil, sebum, and any product build-up from the hair. In some cases, the detoxifying agents may clean the scalp of dandruff, acne, or scabbing. In some cases, the detoxifying agents stimulate hair follicles to allow for hair growth. In some cases, the detoxifying agents reduce itchiness, oiliness, or inflammation. In some embodiments, the detoxifying agent is an absorbent. In some embodiments, the absorbent is a charcoal powder.

In some embodiments, the concentration (% w/w) of the one or more detoxifying agents in the detoxifying hair and scalp cleanser composition is between about 0.01% to about 1.0%. In some embodiments, the concentration of the one or more detoxifying agents in the detoxifying hair and scalp cleanser composition is about 0.01%, about 0.05%, about 0.10%, about 0.15%, about 0.20%, about 0.25%, about 0.30%, about 0.35%, about 0.40%, about 0.45%, about 0.50%, about 0.55%, about 0.60%, about 0.65%, about 0.70%, about 0.75%, about 0.80%, about 0.85%, about 0.90%, about 0.95%, about 1.0%, and any values therebetween. In some embodiments, the concentration of the one or more detoxifying agents in the detoxifying hair and scalp cleanser composition is between about 0.01% to about 0.95%, between about 0.05% to about 1.0%, between about 0.20% to about 0.90%, between about 0.30% to about 0.85%, between about 0.40% to about 0.80%, between about 0.50% to about 0.75%, between about 0.60% to about 0.70%, and between about 0.70% to about 1.0%.

### Cleansing Agents

The detoxifying hair and scalp cleanser composition described herein includes one or more cleansing agents that help to clean the hair but do not remove or replace any essential oils. In some embodiments, the detoxifying hair and scalp cleanser composition comprises one or more cleansing agents selected from: an anionic surfactant, an amphoteric surfactant, a non-ionic surfactant, a biosurfactant, and any combinations thereof.

In some embodiments, the one or more cleansing agents is an anionic surfactant selected from: sodium methyl 2-sulfolaurate, sodium lauryl sulfoacetate, disodium 2-sulfolaurate, alkylbenzene sulfonates, ammonium lauryl sulfate, ammonium xylenesulfonate, sodium laureth sulfate, sodium lauryl sulfate, sodium lauryl sulfoacetate, sodium 2-sulfolaurate, sodium myreth sulfate, sodium xylenesulfonate, sodium C₁₄₋₁₆ olefin sulfonate, sodium C₁₄₋₁₆ alpha olefin sulfonate, disodium lauryl sulfosuccinate, sodium methyl cocoyl taurate, sodium cocoyl isethionate, sodium lauryl isethionate, sodium cocoyl sarcosinate, sodium lauroyl sarcosinate, sodium myristoyl sarcosinate, lauroyl sarcosine, cocoyl sarcosine, myristoyl sarcosine, oleoyl sarcosine, myristoyl/stearoyl sarcosine, sodium lauroyl glutamate, sodium cocoyl glutamate, disodium cocoyl glutamate, cocoyl glutamic acid, lauroyl glutamic acid, potassium cocoyl glutamate, potassium lauroyl glutamate, potassium myristoyl glutamate, sodium myristoyl glutamate, sodium stearoyl glutamate, disodium stearoyl glutamate, sodium palmoyl glutamate, sodium lauroyl lactylate, sodium astrocaryum murumuruate, sodium laurylglucosides hydroxypropylsulfonate, disodium laurylglucosides hydroxypropyl citrate, sodium hydroxypropylsulfonate laurylglucoside crosspolymer, dioctyl sodium sulfosuccinate, and any combinations thereof.

In some embodiments, the one or more cleansing agents is an amphoteric surfactant selected from: cocamindopropyl hydroxysultaine, cocamidopropyl betaine, coco betaine, betaine, cocoamphoacetate, cocoamphodipropionate, disodium cocoamphodiacetate, disodium cocoamphodipropionate, lauryl betaine, lauramidopropyl betaine, lauramidopropyl hydroxysultaine, lauryl hydroxysultaine, sodium dilauramidoglutamide lysine, sodium cocoamphopropionate, lauroamphoacetate, and any combinations thereof.

In some embodiments, the one or more cleansing agents is a non-ionic surfactant selected from: decyl glucoside, lauryl glucoside, coco-glucoside, cocoyl methyl glucamide, capryloyl/caproyl methyl glucamide, lauroyl/myristoyl methyl glucamide, cocamidopropylamine oxide, decylamine oxide, and combinations thereof.

In some embodiments, the one or more cleansing agents is a biosurfactant selected from: Sapindus mukorossi (soapnut) extract, saponin, lipopeptide, potassium cocoyl hydrolyzed oat protein, starmella bombicola/Brassica oil ferment, glycoside, glycolipid, rhamnolipid, phospholipid, and any combinations thereof.

In some embodiments, the concentration (% w/w) of the one or more cleansing agents in the detoxifying hair and scalp cleanser composition is between about 0.1% to about 20%. In some embodiments, the concentration of the one or more cleansing agents in the detoxifying hair and scalp cleanser composition is about 0.1%, about 0.2%, about 0.3%, about 0.4%, about 0.5%, about 0.6%, 0.7%, about 0.8%, about 0.9%, about 1.0%, about 1.2%, about 1.4%, about 1.6%, about 1.8%, about 2.0%, about 2.5%, about 3.0%, about 3.5%, about 4.0%, about 4.5%, about 5.0%, about 5.5%, about 6.0%, about 6.5%, about 7.0%, about 7.5%, about 8.0%, about 8.5%, about 9.0%, about 9.5%, about 10.0%, about 10.5%, about 11.0%, about 11.5%, about 12.0%, about 12.5%, about 13%, about 13.5%, about 14%, about 14.5%, about 15%, about 16%, about 17%, about 18%, about 19%, about 20%, and any values therebetween. In some embodiments, the concentration of the one or more cleansing agents in the detoxifying hair and scalp cleanser composition is between about 0.1% to about 20%, between about 0.2% to about 19%, between about 0.3% to about 18%, between about 0.4% to about 17%, between about 0.5% to about 16%, between about 0.6% to about 15%, between about 0.7% to about 14.5%, between about 0.8% to about 14%, between about 0.9% to about 13.5%, between about 1.0% to about 13%, between about 1.5% to about 12.5%, between about 2.0% to about 12%, between about 2.5% to about 11.5%, between about 3.0% to about 11%, between about 3.5% to about 10.5%, and between about 4% to about 10%.

### Excipients

The detoxifying hair and scalp cleanser composition described herein includes one or more cosmetically or dermatologically acceptable excipients. The cosmetically or dermatologically acceptable excipients includes, but are not limited to, preservatives, antioxidants, thickeners, chelating agents, co-solvents, sunscreen agents, vitamins, dyes, hair coloring agents, proteins, amino acids, organic polymers, natural extracts such as plant extracts, humectants, fragrances, perfumes, oils, emollients, lubricants, butters, penetrants, thickeners, viscosity modifiers, polymers, resins, hair fixatives, silicones, film formers, surfactants, detergents, emulsifiers, opacifying agents, volatiles, propellants, liquid vehicles, carriers, salts, pH adjusting agents (buffers), neutralizing agents, buffers, hair conditioning agents, anti-static agents, anti-frizz agents, anti-dandruff agents, absorbents, and any combinations thereof.

In some embodiments, the concentration (% w/w) of the one or more cosmetically or dermatologically acceptable excipients in the detoxifying hair and scalp cleanser composition is between about 0.1% to about 30%. In some embodiments, the concentration of the one or more cosmetically or dermatologically acceptable excipients in the detoxifying hair and scalp cleanser composition is about 0.1%, about 0.5%, about 1.0%, about 2.0%, about 3.0%, about 4.0%, about 5.0%, about 6.0%, about 7.0%, about 8.0%, about 9.0%, about 10.0%, about 11.0%, about 12.0%, about 13.0%, about 14.0%, about 15.0%, about 16.0%, about 17.0%, about 18.0%, about 19.0%, about 20.0%, about 21.0%, about 22.0%, about 23.0%, about 24.0%, about 25.0%, about 26.0%, about 27.0%, about 28.0%, about 29.0%, about 30.0%, and any values therebetween. In some embodiments, the concentration of the one or more cosmetically or dermatologically acceptable excipients in the detoxifying hair and scalp cleanser composition is between about 0.1% to about 29.0%, between about 0.5% to about 28.0%, between about 1.0% to about 27.0%, between about 2.0% to about 26.0%, between about 3.0% to about 25.0%, between about 4.0% to about 24.0%, between about 5.0% to about 23.0%, between about 6.0% to about 22.0%, between about 7.0% to about 21.0%, between about 8.0% to about 20.0%, between about 9.0% to about 19.0%, between about 10.0% to about 18.0%, between about 11.0% to about 17.0%, between about 12.0% to about 16.0%, between about 13.0% to about 15.0%, between about 14.0% to about 14.5%, and between about 14.5% to about 30.0%.

In some embodiments, the one or more cosmetically or dermatologically acceptable excipients is an emulsifier selected from: caprylic/capric/diglyceryl succinate, C14-16 glycol palmitate, C18-20 glycol isostearate, cocamidopropyl lauryl ether, dipentaerythrityl hydroxystearate, dipentaerythrityl hydroxyisostearate, dipentaerythrityl hexacaprate/caprylate, palm kernel glycerides, lecithin, hydrogenated lecithin, phosphatidylcholine, C12-16 alcohols, palmitic acid, glyceryl behenate, glyceryl stearate, glyceryl stearate citrate, glyceryl oleate citrate, sorbitan laurate, lauryl laurate, sorbitan olivate, cetearyl olivate, glyceryl stearate citrate, sodium stearoyl lactylate, sodium stearoyl glutamate, sodium cocoyl glutamate, caprylyl/capryl glucoside, polyhydroxystearic acid, phenethyl alcohol, sodium surfactin, polyglycerin-3, polyglyceryl-3 distearate, polyglyceryl-3 diisostearate, polyglyceryl-3 cocoate, polyglyceryl-3 dicitrate/stearate, polyglyceryl-3 beeswax, polyglyceryl-3 polyricinoleate, polyglyceryl-4 oleate, polyglyceryl-4 caprate, polylgylceryl-4 laurate, polyglyceryl-6 caprylate, polyglyceryl-6 stearate, polyglyceryl-6 distearate, polyglyceryl-6 behenate, polyglyceryl-6 polyhydroxystearate, polyglyceryl-6 oleate, polyglyceryl-6 ricinoleate, polyglyceryl-6 polyricinoleate, polyglyceryl-10 laurate, candelilla/jojoba/rice bran polyglyceryl-3 esters, stearoyl glutamic acid, Polysorbate-20, polyglyceryl-10 laurate, polyglyceryl-6 caprylate, polyglyceryl-3 cocoate, polyglyceryl-4 caprate, polyglyceryl-6 ricinoleate, polyglyceryl-6 caprylate, sodium surfactin, and any combinations thereof.

Additional chelating molecules can improve stabilization of surfactant formulation foam during the user experience and may even remove some metals from the hair itself, which can lead to softer-feeling hair without the use of conditioning agents. To avoid lack of use and ensuing decrease in applied peptide benefits, the clarifying formula must avoid the consumer-associated perception of unpleasant “stripping”—this can be achieved by the combination of peptides and salicylic acid in formulation to both minimize swelling of the cuticle that could lead to more prominent scales, tangling, breakage, etc. and prevent protein loss during the more intensely clarifying shampoo process. In some embodiments, the one or more cosmetically or dermatologically acceptable excipients is a chelating agent selected from: phytic acid, sodium phytate, caprylhydroxamic acid, trisodium dicarboxymethyl alaninate, gluconic acid, sodium gluconate, pentasodium pentetate, sodium carboxymethyl inulin, tetrasodium glutamate diacetate, trisodium ethylenediamine succinate, sodium thiosulfate, diiospropyl oxalate, disodium EDTA, disodium EDTA-copper, HEDTA, oxalic acid, potassium citrate, sodium citrate, dodium oxalate, TEA-EDTA, tetrasodium EDTA, trisodium EDTA, trisodium HEDTA, and any combinations thereof.

In some embodiments, the one or more cosmetically or dermatologically acceptable excipients is a preservative selected from: benzoic acid, butyl paraben, diazolidinyl urea, DMDM hydantoin, ethyl paraben, imidazolidinyl urea, iodopropynyl butylcarbamate, isobutyl paraben, methyl paraben, methylchloroisothiazolinone, methylisothiazolinone, phenoxyethanol, propyl paraben, sodium benzoate, potassium sorbate, ethylhexylglycerin, gluconolactone, and any combinations thereof.

In some embodiments, the one or more cosmetically or dermatologically acceptable excipients is a thickener selected from: lauryl lactate, hydroxypropyl starch phosphate, acrylates copolymer, ammonium acryloyldimethyltaurate, sodium acryloyldimethyltaurate/VP copolymer, sodium acrylate/sodium acryloyldimethyl taurate copolymer, sodium polyacrylate, polyacrylate crosspolymer-11, carbomer, acrylates/C10-30 alkyl acrylate crosspolymer, hydroxyethyl acrylate/sodiumacryloyldimethyltaurate copolymer, lithium magnesium sodium silicate, sodium magnesium fluorosilicate, glycol distearate, hydrogenated castor oil, hydrogenated castor oil hydroxystearate, hydrogenated castor oil isostearate, hydrogenated castor oil stearate, hydrogenated castor PEG-8 esters, and any combinations thereof.

In some embodiments, the one or more cosmetically or dermatologically acceptable excipients is an anti-static agent such. In some embodiments, the anti-static agent is a cationic surfactant selected from: quaternary ammonium compounds, behentrimonium chloride, behentrimonium methosulfate, cetrimonium chloride, cetrimonium methosulfate, benzalkonium chloride, benzethonium chloride, cinnamidopropyltrimonium chloride, cocotrimonium chloride, dicetyldimonium chloride, dicocodimonium chloride, laurtrimonium chloride, stearamidopropyl dimethylamine, steartrimonium chloride, stearalkonium chloride, tricetyldimonium chloride, PCA ethyl cocoyl arginate, C10-40 isoalkylamidopropylethyldimonium ethosulfate, dipalmitoylethyl hydroxyethylmonium methosulfate, shea butteramidopropyltrimonium chloride, and any combinations thereof.

In some embodiments, the one or more cosmetically or dermatologically acceptable excipients is a naturally derived polymer selected from: xanthan gum, pullulan, algin, tetragonoloba (guar) gum, sclerotium gum, acacia Senegal gum, cellulose, cellulose gum, carboxymethylcellulose, carbomer, hydroxypropyl starch phosphate, guar hydroxypropyltrimonium chloride, carboxymethyl hydroxyethyl cellulose, carboxymethyl hydroxypropyl guar, cellulose, ethyl cellulose, hydroxybutyl methylcellulose, hydroxyethylcellulose, hydroxymethylcellulose, lauryl polyglucose, and any combinations thereof.

In some embodiments, the one or more cosmetically or dermatologically acceptable excipients is an amino acid such as L-Cysteine.

In some embodiments, the one or more cosmetically or dermatologically acceptable excipients is a humectant selected from: 1,2,6 hexanetriol, dipropylene glycol, glycerin, butylene glycol, caprylyl glycol, pentylene glycol, isopentyldiol, hexylene glycol, panthenol, phytantriol, propylene glycol, propanediol, sodium PCA, sorbitol, triethylene glycol, polyglyceryl sorbitol, glucose, fructose, mannitol, xylitol, inositol, trehalose, polydextrose, potassium PCA, hydrogenated honey, hyaluronic acid, inositol, erythritol, capryl glycol, acetamidoethoxyethanol, and any combinations thereof.

In some embodiments, the one or more cosmetically or dermatologically acceptable excipients is a cationic polymer selected from: polyquaternium-10, polyquaternium-7, polyquaternium-11, polyquaternium-57, quaternium-15, quaternium-18, quaternium-22, quaternium-87, guar hydroxypropyltrimonium chloride, and any combinations thereof.

In some embodiments, the one or more cosmetically or dermatologically acceptable excipients is a non-ionic surfactant selected from: decyl glucoside, lauryl glucoside, coco-glucoside, cocoyl methyl glucamide, capryloyl/caproyl methyl glucamide, lauroyl/myristoyl methyl glucamide, cocamidopropylamine oxide, decylamine oxide, and combinations thereof. In some embodiments, the non-ionic surfactant is a solubilizer, emulsifier, stabilizer, emollient, thickener, and any combinations thereof.

In some embodiments, the one or more cosmetically or dermatologically acceptable excipients is a silicone selected from: amodimethicone, amodimethicone, dimethicone cyclomethicone, cyclopentasiloxane, dimethicone, dimethicone copolyol, dimethiconol, hydrolyzed wheat protein hydroxypropyl polysiloxane, trimethylsilylamodimethicone, and any combinations thereof.

In some embodiments, the one or more cosmetically or dermatologically acceptable excipients is a protein selected from: cocodimonium hydroxypropyl hydrolyzed casein, cocodimonium hydroxypropyl hydrolyzed collagen, cocodimonium hydroxypropyl hydrolyzed hair keratin, cocodimonium hydroxypropyl hydrolyzed keratin, cocodimonium hydroxypropyl hydrolyzed rice protein, cocodimonium hydroxypropyl hydrolyzed silk, cocodimonium hydroxypropyl hydrolyzed soy protein, cocodimonium hydroxypropyl hydrolyzed wheat protein, cocodimonium hydroxypropyl silk amino acids, cocoyl hydrolyzed collagen, cocoyl hydrolyzed keratin, hydrolyzed keratin, hydrolyzed oat flour, hydrolyzed silk, hydrolyzed silk protein, hydrolyzed soy protein, hydrolyzed wheat protein, hydrolyzed quinoa, hydrolyzed elastin, hydrolyzed collagen, keratin, potassium cocoyl hydrolyzed collagen, and any combinations thereof.

In some embodiments, the one or more cosmetically or dermatologically acceptable excipients is a vitamin selected from: retinol, retinyl palmitate, ascorbic acid, tocopherol, tocopherol acetate, and any combinations thereof.

In some embodiments, the one or more cosmetically or dermatologically acceptable excipients is a natural extract selected from: Aloe barbadensis leaf extract, Yucca schidigera leaf extract, Yucca schidigera root extract, Camellia sinensis leaf extract, Equisetum arvense leaf extract, Hamamelis virginiana leaf extract, Macrocystis pyrifera kelp extract, and combinations thereof.

In some embodiments, the one or more cosmetically or dermatologically acceptable excipients is an emollient selected from: isopropyl lauroyl sarcosinate, phenethyl benzoate, C12-15 alkyl benzoate, caprylic/capric triglyceride, cetyl octanoate, octyldodecanol, dilinoleic acid/propanediol copolymer, isoamyl laurate, oleic acid, isohexadecane, squalane, isopropyl palmitate, polycitronellol, diheptyl succinate, capryloyl glycerin/sebacic acid copolymer, PPG-3 benzyl ether ethylhexanoate, hydrogenated vegetable oil, triheptanoin, diisopropyl sebacate, diisopropyl adipate, propylene glycol dibenzoate, propoxytetramethyl piperidinyl dimethicone, castor oil/IPDI copolymer, butylene glycol cocoate, butylene glycol dicaprylate/dicaprate (dispersant), propylene glycol dicaprylate/dicaprate, triolein (algae oil), propylene glycol dicaprylate/dicaprate, phytosteryl/octyldodecyl lauroyl glutamate, C10-18 triglycerides, isostearyl alcohol, diisostearyl malate, isoamyl laurate, *isoamyl cocoate, hemisqualane, polycitronellol acetate, ethyl macadamiate, coco-caprylate/caprate, coconut alkanes, C9-12 alkane, C13-15 alkane, C13-16 isoalkane, neopentyl glycol diheptanoate, hydrogenated ethylhexyl olivate, hydrogenated olive oil unsaponifiables, undecane, tridecane, isododecane, caprylyl caprylate/caprate, cetyl stearate, cetearyl stearate, decyl oleate, glyceryl stearate, glyceryl adipate, glyceryl arachidate, glyceryl arachidonate, glyceryl behenate, glyceryl caprate, glyceryl caprylate, glyceryl caprylate/caprate, glyceryl citrate/lactate/linoleate/oleate, glyceryl cocoate, glyceryl diarachidate, glyceryl dibehenate, glyceryl dierucate, glyceryl dihydroxystearate, glyceryl diisopalmitate, glyceryl diisostearate, glyceryl dilaurate, glyceryl dilinoleate, glyceryl dimyristate, glyceryl dioleate, glyceryl dipalmitate, glyceryl dipalmitoleate, glyceryl diricinoleate, glyceryl distearate, glyceryl erucate, glycol stearate, isocetyl stearate, isopropyl myristate, isopropyl palmitate, isopropyl stearate, isostearyl stearate, octyl palmitate, octyl stearate, propylene glycol dicaprylate/dicaprate, sorbitan benzoate, sorbitan caprylate, sorbitan isostearate, sorbitan laurate, sorbitan tristearate, stearyl stearate, tocopheryl linoleate, olive oil, avocado oil, coconut oil, sunflower oil, jojoba oil, argan oil, castor oil, almond oil, Sesamum indicum (sesame) seed oil, Prunus areniaca (apricot) kernel oil, Crambe abyssinica seed oil, Oryza sativa (rice) bran oil, Vitis vinifera (grape) seed oil, Passiflora edulis seed oil, meadowfoam seed oil, Butyrospermum parkii (shea) butter, Shorea robusta seed butter, murumuru butter, and any combinations thereof.

In some embodiments, the one or more cosmetically or dermatologically acceptable excipients is an antioxidant selected from: carnosine, tetrahexyldecyl ascorbate, bioflavonoids, lycopene, Daucus carota sativa root cell culture lysate, monoammonium glycyrrhizinate, alpha lipoic acid (thioctic acid), Leontopodium alpinum (edelweiss) extract, Rosmarinus officinalis (rosemary) leaf extract (WS), Vitis vinifera (grape) seed extract, Camellia sinensis leaf extract, Quercus robur wood extract, hydrolyzed olive fruit, Olea europea (olive) leaf extract, oleuropin, Punica granatum bark/fruit extract, punicalagin, ellagic acid, polyphenols, epigallocatechin (EGCG), tannins, sulforaphane, resveratrol, nordihydroguaiaretic acid, and any combinations thereof.

In some embodiments, the one or more cosmetically or dermatologically acceptable excipients is a buffer selected from: ascorbic acid, citric acid, lactic acid, glycolic acid, tartaric acid, malic acid, L-arginine, potassium hydroxide, sodium hydroxide, triethanolamine, sodium bicarbonate, and any combinations thereof.

In some embodiments, the one or more cosmetically or dermatologically acceptable excipients is a salt selected from: calcium chloride, magnesium chloride, magnesium sulfate, potassium chloride, potassium glycol sulfate, sodium chloride, and any combinations thereof.

In some embodiments, the one or more cosmetically or dermatologically acceptable excipients is an aliphatic alcohol selected from: lauric acid, palmitic acid, stearic acid, arachidic alcohol, behenyl alcohol, cetearyl alcohol, cetyl alcohol, isocetyl alcohol, isostearyl alcohol, lauryl alcohol, myristyl alcohol, stearyl alcohol, C30-50 alcohols, lanolin alcohol, and any combinations thereof.

In some embodiments, the one or more cosmetically or dermatologically acceptable excipients is a perfuming ingredient. In some embodiments, the perfuming ingredient includes a fragrance. In some embodiments, the fragrance includes limonene.

### Solvents

The detoxifying hair and scalp cleanser composition includes one or more solvents. In some embodiments, the one or more solvents is selected from: water, dimethyl isosorbide, triethyl citrate, phenethyl benzoate, dicaprylyl maleate, dilinoleic acid/propanediol copolymer, lower alcohols containing from 1 to 6 carbon atoms, such as ethanol; polyols such as glycerine, glycols such as butylene glycol, isoprene glycol, propylene glycol, dipropylene glycol, propanediol, isopentyldiol, polyethylene glycols such as PEG-8, sorbitol, sugars such as glucose, fructose, maltose, lactose, sucrose; and any combinations thereof.

In some embodiments, the concentration (% w/w) of the one or more solvents or co-solvents in the detoxifying hair and scalp cleanser composition is between about 50% to about 90%. In some embodiments, the concentration of the one or more solvents in the detoxifying hair and scalp cleanser composition is about 50%, about 55%, about 60%, about 65%, 70%, about 75%, about 80%, about 85%, about 90%, and any values therebetween. In some embodiments, the concentration of the one or more solvents in the detoxifying hair and scalp cleanser composition is between about 50% to about 90%, between about 55% to about 85%, between about 60% to about 80%, between about 65% to about 80%, and between about 70% to about 80%.

### Formulations

In some embodiments, the hair cleanser composition comprises: one or more solvents;
one or more peptides;
one or more hair conditioners;
one or more skin conditioners;
one or more detoxifying agents;
one or more cleansing agents; and
one or more cosmetically or dermatologically acceptable excipients;
wherein the one or more peptides comprises a peptide sequence comprising SEQ ID NO: 75, SEQ ID NO: 400, SEQ ID NO: 401, SEQ ID NO: 402, SEQ ID NO: 409, SEQ ID NO: 412, SEQ ID NO: 432, SEQ ID NO:1131, or a combination thereof.

In some embodiments, the hair cleanser composition comprises: between about 50% to about 90% (% w/w) of one or more solvents;
between about 0.0001% to about 1.0% (% w/w) of one or more peptides;
between about 0.01% to about 10.0% (% w/w) of one or more hair conditioners;
between about 0.01% to about 5.0% (% w/w) one or more skin conditioners;
between about 0.01% to about 1.0% (% w/w) one or more detoxifying agents;
between about 0.1% to about 20% (% w/w) one or more cleansing agents; and
between about 0.1% to about 30% (% w/w) one or more cosmetically or dermatologically acceptable excipients;
wherein the one or more peptides comprises a peptide sequence comprising SEQ ID NO: 75, SEQ ID NO: 400, SEQ ID NO: 401, SEQ ID NO: 402, SEQ ID NO: 409, SEQ ID NO: 412, SEQ ID NO: 432, SEQ ID NO:1131, or a combination thereof.

In some embodiments, the hair cleanser composition comprises: between about 55% to about 85% (% w/w) of one or more solvents;
between about 0.0001% to about 0.9% (% w/w) of one or more peptides;
between about 0.20% to about 9% (% w/w) of one or more hair conditioners;
between about 0.01% to about 0.95% (% w/w) one or more skin conditioners;
between about 0.05% to about 1.0% (% w/w) one or more detoxifying agents;
between about 0.3% to about 18% (% w/w) one or more cleansing agents; and between about 0.5% to about 28.0% (% w/w) one or more cosmetically or dermatologically acceptable excipients;
wherein the one or more peptides comprises a peptide sequence comprising SEQ ID NO: 75, SEQ ID NO: 400, SEQ ID NO: 401, SEQ ID NO: 402, SEQ ID NO: 409, SEQ ID NO: 412, SEQ ID NO: 432, SEQ ID NO:1131, or a combination thereof.

In some embodiments, the hair cleanser composition comprises: between about 55% to about 85% (% w/w) of one or more solvents;
between about 0.0001% to about 0.9% (% w/w) of one or more peptides;
between about 0.20% to about 9% (% w/w) of one or more hair conditioners;
between about 0.01% to about 0.95% (% w/w) one or more skin conditioners;
between about 0.05% to about 1.0% (% w/w) one or more detoxifying agents comprising an absorbent;
between about 0.3% to about 18% (% w/w) one or more cleansing agents selected from: an anionic surfactant, an amphoteric surfactant, a non-ionic surfactant, a biosurfactant, and any combinations thereof; and
between about 0.5% to about 28.0% (% w/w) one or more cosmetically or dermatologically acceptable excipients selected from: preservatives, antioxidants, thickeners, chelating agents, co-solvents, sunscreen agents, vitamins, dyes, hair coloring agents, proteins, amino acids, organic polymers, natural extracts such as plant extracts, humectants, fragrances, perfumes, oils, emollients, lubricants, butters, penetrants, thickeners, viscosity modifiers, polymers, resins, hair fixatives, silicones, film formers, surfactants, detergents, emulsifiers, opacifying agents, volatiles, propellants, liquid vehicles, carriers, salts, pH adjusting agents (buffers), neutralizing agents, buffers, hair conditioning agents, anti-static agents, anti-frizz agents, anti-dandruff agents, absorbents, and any combinations thereof;
wherein the one or more peptides comprises a peptide sequence comprising SEQ ID NO: 75, SEQ ID NO: 400, SEQ ID NO: 401, SEQ ID NO: 402, SEQ ID NO: 409, SEQ ID NO: 412, SEQ ID NO: 432, SEQ ID NO:1131, or a combination thereof.

In some embodiments, the hair cleanser composition comprises: between about 55% to about 85% (% w/w) of one or more solvents selected from:
water, dimethyl isosorbide, triethyl citrate, phenethyl benzoate, dicaprylyl maleate, dilinoleic acid/propanediol copolymer, lower alcohols containing from 1 to 6 carbon atoms, such as ethanol; polyols such as glycerine, glycols such as butylene glycol, isoprene glycol, propylene glycol, dipropylene glycol, propanediol, isopentyldiol, polyethylene glycols such as PEG-8, sorbitol, sugars such as glucose, fructose, maltose, lactose, sucrose; and any combinations thereof;
between about 0.0001% to about 0.9% (% w/w) of one or more peptides;
between about 0.20% to about 9% (% w/w) of one or more hair conditioners selected from: panthenol, salicylic acid, sodium salicylate, carnosine, tetrahexyldecyl ascorbate, hyaluronic acid/sodium hyaluronate, hydrolyzed hyaluronic acid, sarcosine, PCA, arginine PCA, brassicyl valinate esylate, brassicyl isoleucinate esylate, brassica alcohol, glycolipids, glycosphingolipids, sphinganine, Rubus ideaus (raspberry) leaf extract, bis-ethyl(isostearylimidazoline) isostearamide, Camellia sinensis leaf extract, Carthamus tinctorius (safflower) seed extract, Glycine soja (soybean) seed extract, Elaeis guineensis (palm) fruit extract, Oryza sativa (rice) extract, hydrolyzed Adansonia digitata seed extract, hydrolyzed corn starch, behenyl/stearyl aminopropanediol esters, C10-40 isoalkylamidopropylethyldimonium ethosulfate, Linum usitatissimum (linseed) extract, Salva hispanica seed extract, Astrocaryum mururmuru fruit extract, Moringa oleifera seed extract, cinnamidopropyltrimonium chloride, hydroxypropyl guar hydroxypropyltrimonium chloride, Trifolium pratense (clover) flower extract, Opuntia ficus-indica stem extract, Kappaphycus alvarezii extract, Laminaria saccharina extract, guar hydroxypropyltriammonium chloride, bis-diglyceryl polyacyladipate-2, hydroxypropylgluconamide, hydroxypropylammonium gluconate, biosaccharide gum-4, prunus Amygdalus dulcic (sweet almond) seed extract, soybean glycerides, Butyrospermum parkii (shea) butter unsaponifiables, hydrolyzed chestnut extract, glyceryl rosinate, sodium methoxy peg-16 maleate/styrene sulfonate copolymer, panthenyl hydroxypropyl steardimonium chloride, and any combinations thereof, between about 0.01% to about 0.95% (% w/w) one or more skin conditioners selected from: niacinamide, allantoin, maltooligosyl glucoside, hydrogenated starch hydrolysate, Opuntia ficus-indica stem extract, sodium hyaluronate crosspolymer, jojoba esters, hydrolyzed jojoba esters, oleic acid, linoleic acid, linolenic acid, lactobionic acid, ursolic acid/sodium ursolate, nordihydroguaiaretic acid, ceramides, fructooligosaccharides, galactoarabinan, schizophyllan, Anetholea anisate leaf extract, Tetraselmis suecica extract, saccharide isomerase, Alteromonas ferment extract, Pseudoalteromonas ferment extract, Corylus avellana leaf extract, Phyllanthus emblica fruit extract, Betula alba juice, Saccharum officinarum (sugarcane) extract, Rhodomyrtus tomentosa fruit extract, Hedychium coronarium root extract, Epilobium angustifolium flower/leaf/stem extract, Tremella fuciformis sporocarp extract, alpha-glucan oligosaccharide, and any combinations thereof;
between about 0.05% to about 1.0% (% w/w) one or more detoxifying agents comprising an absorbent;
between about 0.3% to about 18% (% w/w) one or more cleansing agents selected from: an anionic surfactant, an amphoteric surfactant, a non-ionic surfactant, a biosurfactant, and any combinations thereof; and
between about 0.5% to about 28.0% (% w/w) one or more cosmetically or dermatologically acceptable excipients selected from: preservatives, antioxidants, thickeners, chelating agents, co-solvents, sunscreen agents, vitamins, dyes, hair coloring agents, proteins, amino acids, organic polymers, natural extracts such as plant extracts, humectants, fragrances, perfumes, oils, emollients, lubricants, butters, penetrants, thickeners, viscosity modifiers, polymers, resins, hair fixatives, silicones, film formers, surfactants, detergents, emulsifiers, opacifying agents, volatiles, propellants, liquid vehicles, carriers, salts, pH adjusting agents (buffers), neutralizing agents, buffers, hair conditioning agents, anti-static agents, anti-frizz agents, anti-dandruff agents, absorbents, and any combinations thereof;
wherein the one or more peptides comprises a peptide sequence comprising SEQ ID NO: 75, SEQ ID NO: 400, SEQ ID NO: 401, SEQ ID NO: 402, SEQ ID NO: 409, SEQ ID NO: 412, SEQ ID NO: 432, SEQ ID NO:1131, or a combination thereof.

In some embodiments, the hair cleanser composition comprises:
between about 55% to about 85% (% w/w) of one or more solvents selected from: water, dimethyl isosorbide, triethyl citrate, phenethyl benzoate, dicaprylyl maleate, dilinoleic acid/propanediol copolymer, lower alcohols containing from 1 to 6 carbon atoms, such as ethanol; polyols such as glycerine, glycols such as butylene glycol, isoprene glycol, propylene glycol, dipropylene glycol, propanediol, isopentyldiol, polyethylene glycols such as PEG-8, sorbitol, sugars such as glucose, fructose, maltose, lactose, sucrose; and any combinations thereof;
between about 0.0001% to about 0.9% (% w/w) of one or more peptides;
between about 0.20% to about 9% (% w/w) of one or more hair conditioners selected from: panthenol, salicylic acid, sodium salicylate, carnosine, tetrahexyldecyl ascorbate, hyaluronic acid/sodium hyaluronate, hydrolyzed hyaluronic acid, sarcosine, PCA, arginine PCA, brassicyl valinate esylate, brassicyl isoleucinate esylate, brassica alcohol, glycolipids, glycosphingolipids, sphinganine, Rubus ideaus (raspberry) leaf extract, bis-ethyl(isostearylimidazoline) isostearamide, Camellia sinensis leaf extract, Carthamus tinctorius (safflower) seed extract, Glycine soja (soybean) seed extract, Elaeis guineensis (palm) fruit extract, Oryza sativa (rice) extract, hydrolyzed Adansonia digitata seed extract, hydrolyzed corn starch, behenyl/stearyl aminopropanediol esters, C10-40 isoalkylamidopropylethyldimonium ethosulfate, Linum usitatissimum (linseed) extract, Salva hispanica seed extract, Astrocaryum mururmuru fruit extract, Moringa oleifera seed extract, cinnamidopropyltrimonium chloride, hydroxypropyl guar hydroxypropyltrimonium chloride, Trifolium pratense (clover) flower extract, Opuntia ficus-indica stem extract, Kappaphycus alvarezii extract, Laminaria saccharina extract, guar hydroxypropyltriammonium chloride, bis-diglyceryl polyacyladipate-2, hydroxypropylgluconamide, hydroxypropylammonium gluconate, biosaccharide gum-4, prunus Amygdalus dulcic (sweet almond) seed extract, soybean glycerides, Butyrospermum parkii (shea) butter unsaponifiables, hydrolyzed chestnut extract, glyceryl rosinate, sodium methoxy peg-16 maleate/styrene sulfonate copolymer, panthenyl hydroxypropyl steardimonium chloride, and any combinations thereof,
between about 0.01% to about 0.95% (% w/w) one or more skin conditioners selected from: niacinamide, allantoin, maltooligosyl glucoside, hydrogenated starch hydrolysate, Opuntia ficus-indica stem extract, sodium hyaluronate crosspolymer, jojoba esters, hydrolyzed jojoba esters, oleic acid, linoleic acid, linolenic acid, lactobionic acid, ursolic acid/sodium ursolate, nordihydroguaiaretic acid, ceramides, fructooligosaccharides, galactoarabinan, schizophyllan, Anetholea anisate leaf extract, Tetraselmis suecica extract, saccharide isomerase, Alteromonas ferment extract, Pseudoalteromonas ferment extract, Corylus avellana leaf extract, Phyllanthus emblica fruit extract, Betula alba juice, Saccharum officinarum (sugarcane) extract, Rhodomyrtus tomentosa fruit extract, Hedychium coronarium root extract, Epilobium angustifolium flower/leaf/stem extract, Tremella fuciformis sporocarp extract, alpha-glucan oligosaccharide, and any combinations thereof;
between about 0.05% to about 1.0% (% w/w) one or more detoxifying agents comprising charcoal powder;
between about 0.3% to about 18% (% w/w) one or more cleansing agents selected from: sodium methyl 2-sulfolaurate, sodium lauryl sulfoacetate, disodium 2-sulfolaurate, alkylbenzene sulfonates, ammonium lauryl sulfate, ammonium xylenesulfonate, sodium laureth sulfate, sodium lauryl sulfate, sodium lauryl sulfoacetate, sodium 2-sulfolaurate, sodium myreth sulfate, sodium xylenesulfonate, sodium C₁₄₋₁₆ olefin sulfonate, sodium C₁₄₋₁₆ alpha olefin sulfonate, disodium lauryl sulfosuccinate, sodium methyl cocoyl taurate, sodium cocoyl isethionate, sodium lauryl isethionate, sodium cocoyl sarcosinate, sodium lauroyl sarcosinate, sodium myristoyl sarcosinate, lauroyl sarcosine, cocoyl sarcosine, myristoyl sarcosine, oleoyl sarcosine, myristoyl/stearoyl sarcosine, sodium lauroyl glutamate, sodium cocoyl glutamate, disodium cocoyl glutamate, cocoyl glutamic acid, lauroyl glutamic acid, potassium cocoyl glutamate, potassium lauroyl glutamate, potassium myristoyl glutamate, sodium myristoyl glutamate, sodium stearoyl glutamate, disodium stearoyl glutamate, sodium palmoyl glutamate, sodium lauroyl lactylate, sodium astrocaryum murumuruate, sodium laurylglucosides hydroxypropylsulfonate, disodium laurylglucosides hydroxypropyl citrate, sodium hydroxypropylsulfonate laurylglucoside crosspolymer, dioctyl sodium sulfosuccinate, cocamindopropyl hydroxysultaine, cocamidopropyl betaine, coco betaine, betaine, cocoamphoacetate, cocoamphodipropionate, disodium cocoamphodiacetate, disodium cocoamphodipropionate, lauryl betaine, lauramidopropyl betaine, lauramidopropyl hydroxysultaine, lauryl hydroxysultaine, sodium dilauramidoglutamide lysine, sodium cocoamphopropionate, lauroamphoacetate, decyl glucoside, lauryl glucoside, coco-glucoside, cocoyl methyl glucamide, capryloyl/caproyl methyl glucamide, lauroyl/myristoyl methyl glucamide, cocamidopropylamine oxide, decylamine oxide, Sapindus mukorossi (soapnut) extract, saponin, lipopeptide, canola sophorolipid, potassium cocoyl hydrolyzed oat protein, starmella bombicola/Brassica oil ferment, glycoside, glycolipid, rhamnolipid, phospholipid, sophorolipid, and any combinations thereof; and
between about 0.5% to about 28.0% (% w/w) one or more cosmetically or dermatologically acceptable excipients selected from: caprylic/capric/diglyceryl succinate, C14-16 glycol palmitate, C18-20 glycol isostearate, cocamidopropyl lauryl ether, dipentaerythrityl hydroxystearate, dipentaerythrityl hydroxyisostearate, dipentaerythrityl hexacaprate/caprylate, palm kernel glycerides, lecithin, hydrogenated lecithin, phosphatidylcholine, C12-16 alcohols, palmitic acid, glyceryl behenate, glyceryl stearate, glyceryl stearate citrate, glyceryl oleate citrate, sorbitan laurate, lauryl laurate, sorbitan olivate, cetearyl olivate, glyceryl stearate citrate, sodium stearoyl lactylate, sodium stearoyl glutamate, sodium cocoyl glutamate, caprylyl/capryl glucoside, polyhydroxystearic acid, phenethyl alcohol, sodium surfactin, polyglycerin-3, polyglyceryl-3 distearate, polyglyceryl-3 diisostearate, polyglyceryl-3 cocoate, polyglyceryl-3 dicitrate/stearate, polyglyceryl-3 beeswax, polyglyceryl-3 polyricinoleate, polyglyceryl-4 oleate, polyglyceryl-4 caprate, polylgylceryl-4 laurate, polyglyceryl-6 caprylate, polyglyceryl-6 stearate, polyglyceryl-6 distearate, polyglyceryl-6 behenate, polyglyceryl-6 polyhydroxystearate, polyglyceryl-6 oleate, polyglyceryl-6 ricinoleate, polyglyceryl-6 polyricinoleate, polyglyceryl-10 laurate, candelilla/jojoba/rice bran polyglyceryl-3 esters, stearoyl glutamic acid, Polysorbate-20, polyglyceryl-10 laurate, polyglyceryl-6 caprylate, polyglyceryl-3 cocoate, polyglyceryl-4 caprate, polyglyceryl-6 ricinoleate, polyglyceryl-6 caprylate, sodium surfactin, phytic acid, sodium phytate, caprylhydroxamic acid, trisodium dicarboxymethyl alaninate, gluconic acid, sodium gluconate, pentasodium pentetate, sodium carboxymethyl inulin, tetrasodium glutamate diacetate, trisodium ethylenediamine succinate, sodium thiosulfate, diiospropyl oxalate, disodium EDTA, disodium EDTA-copper, HEDTA, oxalic acid, potassium citrate, sodium citrate, dodium oxalate, TEA-EDTA, tetrasodium EDTA, trisodium EDTA, trisodium HEDTA, benzoic acid, butyl paraben, diazolidinyl urea, DMDM hydantoin, ethyl paraben, imidazolidinyl urea, iodopropynyl butylcarbamate, isobutyl paraben, methyl paraben, methylchloroisothiazolinone, methylisothiazolinone, phenoxyethanol, propyl paraben, sodium benzoate, potassium sorbate, ethylhexylglycerin, gluconolactone, lauryl lactate, hydroxypropyl starch phosphate, acrylates copolymer, ammonium acryloyldimethyltaurate, sodium acryloyldimethyltaurate/VP copolymer, sodium acrylate/sodium acryloyldimethyl taurate copolymer, sodium polyacrylate, polyacrylate crosspolymer-11, carbomer, acrylates/C10-30 alkyl acrylate crosspolymer, hydroxyethyl acrylate/sodiumacryloyldimethyltaurate copolymer, lithium magnesium sodium silicate, sodium magnesium fluorosilicate, glycol distearate, hydrogenated castor oil, hydrogenated castor oil hydroxystearate, hydrogenated castor oil isostearate, hydrogenated castor oil stearate, hydrogenated castor PEG-8 esters, quaternary ammonium compounds, behentrimonium chloride, behentrimonium methosulfate, cetrimonium chloride, cetrimonium methosulfate, benzalkonium chloride, benzethonium chloride, cinnamidopropyltrimonium chloride, cocotrimonium chloride, dicetyldimonium chloride, dicocodimonium chloride, laurtrimonium chloride, stearamidopropyl dimethylamine, steartrimonium chloride, stearalkonium chloride, tricetyldimonium chloride, PCA ethyl cocoyl arginate, C10-40 isoalkylamidopropylethyldimonium ethosulfate, dipalmitoylethyl hydroxyethylmonium methosulfate, shea butteramidopropyltrimonium chloride, xanthan gum, pullulan, algin, Tetragonoloba (guar) gum, Sclerotium gum, acacia Senegal gum, cellulose, cellulose gum, carboxymethylcellulose, carbomer, hydroxypropyl starch phosphate, guar hydroxypropyltrimonium chloride, carboxymethyl hydroxyethyl cellulose, carboxymethyl hydroxypropyl guar, cellulose, ethyl cellulose, hydroxybutyl methylcellulose, hydroxyethylcellulose, hydroxymethylcellulose, lauryl polyglucose, L-Cysteine, 1,2,6 hexanetriol, dipropylene glycol, glycerin, butylene glycol, caprylyl glycol, pentylene glycol, isopentyldiol, hexylene glycol, panthenol, phytantriol, propylene glycol, propanediol, sodium PCA, sorbitol, triethylene glycol, polyglyceryl sorbitol, glucose, fructose, mannitol, xylitol, inositol, trehalose, polydextrose, potassium PCA, hydrogenated honey, hyaluronic acid, inositol, erythritol, capryl glycol, acetamidoethoxyethanol, polyquaternium-10, polyquaternium-7, polyquaternium-11, polyquaternium-57, quaternium-15, quaternium-18, quaternium-22, quaternium-87, guar hydroxypropyltrimonium chloride, decyl glucoside, lauryl glucoside, coco-glucoside, cocoyl methyl glucamide, capryloyl/caproyl methyl glucamide, lauroyl/myristoyl methyl glucamide, cocamidopropylamine oxide, decylamine oxide, amodimethicone, amodimethicone, dimethicone cyclomethicone, cyclopentasiloxane, dimethicone, dimethicone copolyol, dimethiconol, hydrolyzed wheat protein hydroxypropyl polysiloxane, trimethylsilylamodimethicone, cocodimonium hydroxypropyl hydrolyzed casein, cocodimonium hydroxypropyl hydrolyzed collagen, cocodimonium hydroxypropyl hydrolyzed hair keratin, cocodimonium hydroxypropyl hydrolyzed keratin, cocodimonium hydroxypropyl hydrolyzed rice protein, cocodimonium hydroxypropyl hydrolyzed silk, cocodimonium hydroxypropyl hydrolyzed soy protein, cocodimonium hydroxypropyl hydrolyzed wheat protein, cocodimonium hydroxypropyl silk amino acids, cocoyl hydrolyzed collagen, cocoyl hydrolyzed keratin, hydrolyzed keratin, hydrolyzed oat flour, hydrolyzed silk, hydrolyzed silk protein, hydrolyzed soy protein, hydrolyzed wheat protein, hydrolyzed quinoa, hydrolyzed elastin, hydrolyzed collagen, keratin, potassium cocoyl hydrolyzed collagen, retinol, retinyl palmitate, ascorbic acid, tocopherol, tocopherol acetate, Aloe barbadensis leaf extract, Yucca schidigera leaf extract, Yucca schidigera root extract, Camellia sinensis leaf extract, Equisetum arvense leaf extract, Hamamelis virginiana leaf extract, Macrocystis pyrifera kelp extract, isopropyl lauroyl sarcosinate, phenethyl benzoate, C12-15 alkyl benzoate, caprylic/capric triglyceride, cetyl octanoate, octyldodecanol, dilinoleic acid/propanediol copolymer, isoamyl laurate, oleic acid, isohexadecane, squalane, isopropyl palmitate, polycitronellol, diheptyl succinate, capryloyl glycerin/sebacic acid copolymer, PPG-3 benzyl ether ethylhexanoate, hydrogenated vegetable oil, triheptanoin, diisopropyl sebacate, diisopropyl adipate, propylene glycol dibenzoate, propoxytetramethyl piperidinyl dimethicone, castor oil/IPDI copolymer, butylene glycol cocoate, butylene glycol dicaprylate/dicaprate (dispersant), propylene glycol dicaprylate/dicaprate, triolein (algae oil), propylene glycol dicaprylate/dicaprate, phytosteryl/octyldodecyl lauroyl glutamate, C10-18 triglycerides, isostearyl alcohol, diisostearyl malate, isoamyl laurate, *isoamyl cocoate, hemisqualane, polycitronellol acetate, ethyl macadamiate, coco-caprylate/caprate, coconut alkanes, C9-12 alkane, C13-15 alkane, C13-16 isoalkane, neopentyl glycol diheptanoate, hydrogenated ethylhexyl olivate, hydrogenated olive oil unsaponifiables, undecane, tridecane, isododecane, caprylyl caprylate/caprate, cetyl stearate, cetearyl stearate, decyl oleate, glyceryl stearate, glyceryl adipate, glyceryl arachidate, glyceryl arachidonate, glyceryl behenate, glyceryl caprate, glyceryl caprylate, glyceryl caprylate/caprate, glyceryl citrate/lactate/linoleate/oleate, glyceryl cocoate, glyceryl diarachidate, glyceryl dibehenate, glyceryl dierucate, glyceryl dihydroxystearate, glyceryl diisopalmitate, glyceryl diisostearate, glyceryl dilaurate, glyceryl dilinoleate, glyceryl dimyristate, glyceryl dioleate, glyceryl dipalmitate, glyceryl dipalmitoleate, glyceryl diricinoleate, glyceryl distearate, glyceryl erucate, glycol stearate, isocetyl stearate, isopropyl myristate, isopropyl palmitate, isopropyl stearate, isostearyl stearate, octyl palmitate, octyl stearate, propylene glycol dicaprylate/dicaprate, sorbitan benzoate, sorbitan caprylate, sorbitan isostearate, sorbitan laurate, sorbitan tristearate, stearyl stearate, tocopheryl linoleate, olive oil, avocado oil, coconut oil, sunflower oil, jojoba oil, argan oil, castor oil, almond oil, Sesamum indicum (sesame) seed oil, prunus areniaca (apricot) kernel oil, Crambe abyssinica seed oil, Oryza sativa (rice) bran oil, Vitis vinifera (grape) seed oil, Passiflora edulis seed oil, meadowfoam seed oil, Butyrospermum parkii (shea) butter, Shorea robusta seed butter, murumuru butter, carnosine, tetrahexyldecyl ascorbate, bioflavonoids, lycopene, Daucus carota sativa root cell culture lysate, monoammonium glycyrrhizinate, alpha lipoic acid (thioctic acid), Leontopodium alpinum (edelweiss) extract, Rosmarinus officinalis (rosemary) leaf extract (WS), Vitis vinifera (grape) seed extract, Camellia sinensis leaf extract, Quercus robur wood extract, hydrolyzed olive fruit, Olea europea (olive) leaf extract, oleuropin, Punica granatum bark/fruit extract, punicalagin, ellagic acid, polyphenols, epigallocatechin (EGCG), tannins, sulforaphane, resveratrol, nordihydroguaiaretic acid, ascorbic acid, citric acid, lactic acid, glycolic acid, tartaric acid, malic acid, L-arginine, potassium hydroxide, sodium hydroxide, triethanolamine, sodium bicarbonate, calcium chloride, magnesium chloride, magnesium sulfate, potassium chloride, potassium glycol sulfate, sodium chloride, lauric acid, palmitic acid, stearic acid, arachidic alcohol, behenyl alcohol, cetearyl alcohol, cetyl alcohol, isocetyl alcohol, isostearyl alcohol, lauryl alcohol, myristyl alcohol, stearyl alcohol, C30-50 alcohols, lanolin alcohol, and any combination thereof,
wherein the one or more peptides comprises a peptide sequence comprising SEQ ID NO: 75, SEQ ID NO: 400, SEQ ID NO: 401, SEQ ID NO: 402, SEQ ID NO: 409, SEQ ID NO: 412, SEQ ID NO: 432, SEQ ID NO:1131, or a combination thereof.

### EXAMPLES

The following examples are included for illustrative purposes only and are not intended to limit the scope of the invention.

### Example 1. Preparation of Detoxifying Hair and Scalp Cleanser

An exemplary detoxifying hair and scalp cleanser composition is provided in Tables 2-6. The exemplary detoxifying hair and scalp cleanser compositions are made by conventional formulation and mixing techniques or by mixing together one or more polyols (e.g. glycerin.), water and surfactants along with any solids that need to be melted at an elevated temperature. The ingredients are mixed thoroughly at the elevated temperature and then cooled to ambient temperature. Additional ingredients are added to the cooled product. The amount stated reflects the weight percent of the active material, unless otherwise specified.

TABLE 2
Exemplary detoxifying hair and scalp cleanser composition

| | | Percent |
|---|---|---|
| Ingredient | Function | formula (% w/w) |
| Water (Aqua) (Eau) | Solvent | 76.3328 |
| Sodium C₁₄₋₁₆ Olefin Sulfonate | Surfactant | 15.44 |
| Cocamidopropyl Hydroxysultaine | Surfactant | 4.49 |
| Salicylic Acid | Hair Conditioning | 1.0 |
| Caprylyl Glycol | Skin Conditioning | 0.75 |
| Citric Acid | Buffering | 0.31 |
| Sodium Phytate | Chelating | 0.30 |
| Guar Hydroxypropyltrimonium | Skin Conditioning | 0.25 |
| Chloride | | |
| Panthenol | Hair Conditioning | 0.20 |
| Glycerin | Humectant | 0.15 |
| Caprylhydroxamic Acid | Chelating | 0.10 |
| Charcoal Powder | Absorbent | 0.06 |
| Potassium Hydroxide | Buffering | 0.01 |
| SEQ ID 409 | Peptide | 0.0001 |
| Fragrance (Parfum) | Perfuming | 0.45414 |
| Limonene | Perfuming | 0.134526 |
| Benzyl Salicylate | Perfuming | 0.011334 |

TABLE 3
Exemplary detoxifying hair and scalp cleanser composition

| | | Percent |
|---|---|---|
| Ingredient | Function | formula (% w/w) |
| Water (Aqua) (Eau) | Solvent | 78.6699 |
| Sodium C₁₄₋₁₆ Olefin Sulfonate | Surfactant | 13.65 |
| Cocamidopropyl Hydroxysultaine | Surfactant | 3.95 |
| Salicylic Acid | Hair Conditioning | 1.0 |
| Caprylyl Glycol | Skin Conditioning | 0.75 |
| Citric Acid | Buffering | 0.31 |
| Sodium Phytate | Chelating | 0.30 |
| Guar Hydroxypropyltrimonium | Skin Conditioning | 0.25 |
| Chloride | | |
| Panthenol | Hair Conditioning | 0.20 |
| Glycerin | Humectant | 0.15 |
| Caprylhydroxamic Acid | Chelating | 0.10 |
| Charcoal Powder | Absorbent | 0.06 |
| Potassium Hydroxide | Buffering | 0.01 |
| SEQ ID 400 | Peptide | 0.0001 |
| Fragrance (Parfum) | Perfuming | 0.45414 |
| Limonene | Perfuming | 0.134526 |
| Benzyl Salicylate | Perfuming | 0.011334 |

TABLE 4
Exemplary detoxifying hair and scalp cleanser composition

| | | Percent |
|---|---|---|
| Ingredient | Function | formula (% w/w) |
| Water (Aqua) (Eau) | Solvent | 80.7684 |
| Sodium C₁₄₋₁₆ Olefin Sulfonate | Surfactant | 11.89 |
| Cocamidopropyl Hydroxysultaine | Surfactant | 3.62 |
| Salicylic Acid | Hair Conditioning | 1.0 |
| Caprylyl Glycol | Skin Conditioning | 0.75 |
| Citric Acid | Buffering | 0.31 |
| Sodium Phytate | Chelating | 0.30 |
| Guar Hydroxypropyltrimonium | Skin Conditioning | 0.25 |
| Chloride | | |
| Panthenol | Hair Conditioning | 0.20 |
| Glycerin | Humectant | 0.15 |
| Caprylhydroxamic Acid | Chelating | 0.10 |
| Charcoal Powder | Absorbent | 0.06 |
| Potassium Hydroxide | Buffering | 0.01 |
| SEQ ID 412 | Peptide | 0.00012 |
| Fragrance (Parfum) | Perfuming | 0.45414 |
| Limonene | Perfuming | 0.134526 |
| Benzyl Salicylate | Perfuming | 0.011334 |

TABLE 5
Exemplary detoxifying hair and scalp cleanser composition

| | | Percent |
|---|---|---|
| Ingredient | Function | formula (% w/w) |
| Water (Aqua) (Eau) | Solvent | 82.1115 |
| Sodium C₁₄₋₁₆ Olefin Sulfonate | Surfactant | 10.6345 |
| Cocamidopropyl Hydroxysultaine | Surfactant | 3.5243 |
| Salicylic Acid | Hair Conditioning | 0.9999 |
| Caprylyl Glycol | Skin Conditioning | 0.75 |
| Citric Acid | Buffering | 0.30 |
| Sodium Phytate | Chelating | 0.2999 |
| Guar Hydroxypropyltrimonium | Skin Conditioning | 0.214 |
| Chloride | | |
| Panthenol | Hair Conditioning | 0.21 |
| Glycerin | Humectant | 0.16 |
| Caprylhydroxamic Acid | Chelating | 0.09 |
| Charcoal Powder | Absorbent | 0.07 |
| Potassium Hydroxide | Buffering | 0.02 |
| SEQ ID 432 | Peptide | 0.00012 |
| Fragrance (Parfum) | Perfuming | 0.456111 |
| Limonene | Perfuming | 0.145678 |
| Benzyl Salicylate | Perfuming | 0.013999 |

TABLE 6
Exemplary detoxifying hair and scalp cleanser composition

| | | Percent |
|---|---|---|
| Ingredient | Function | formula (% w/w) |
| Water (Aqua) (Eau) | Solvent | 83.1119 |
| Sodium C₁₄₋₁₆ Olefin Sulfonate | Surfactant | 12.3989 |
| Cocamidopropyl Hydroxysultaine | Surfactant | 3.555 |
| Salicylic Acid | Hair Conditioning | 1.1 |
| Caprylyl Glycol | Skin Conditioning | 0.78 |
| Citric Acid | Buffering | 0.33 |
| Sodium Phytate | Chelating | 0.29 |
| Guar Hydroxypropyltrimonium | Skin Conditioning | 0.24 |
| Chloride | | |
| Panthenol | Hair Conditioning | 0.21 |
| Glycerin | Humectant | 0.16 |
| Caprylhydroxamic Acid | Chelating | 0.11 |
| Charcoal Powder | Absorbent | 0.08 |
| Potassium Hydroxide | Buffering | 0.02 |
| SEQ ID 401 | Peptide | 0.00014 |
| Fragrance (Parfum) | Perfuming | 0.4897 |
| Limonene | Perfuming | 0.127583 |
| Benzyl Salicylate | Perfuming | 0.012675 |

### Example 2. Evaluations of Detoxifying Hair and Scalp Cleanser Efficacy During and After Cleansing

A detoxifying hair and scalp cleanser composition is prepared according to Example 1. The detoxifying hair and scalp cleanser composition is evaluated using a survey to determine the sensory effect of the detoxifying hair and scalp cleanser on the glossiness, texture (i.e., slipperiness), elasticity and stiffness. The sensory effect is evaluated during washing and after drying.

A human hair bundle having a length of 25 cm, a width of 5.5 cm, and a weight of 10 g is lightly rinsed with warm water at 40° C. Excess water is removed, and the hair bundle is foamed thoroughly for about 1 minute using 1.5 g of the detoxifying hair and scalp cleanser composition per 10-gram tress of detoxifying hair and scalp cleanser. A sensory evaluation is performed on the foamed hair to determine its slipperiness. The evaluation is performed by 20 people, and the total value of the evaluation is shown. Evaluation criteria are 4: glide well, 3: slightly slip, 2: does not slip very well, and 1: do not slip.

A human hair bundle having a length of 25 cm, a width of 5.5 cm, and a weight of 10 g is lightly rinsed with warm water at 40° C. Excess water is removed, and the hair bundle is foamed thoroughly for about 1 minute using 1.5 g of the detoxifying hair and scalp cleanser composition per 10-gram tress of detoxifying hair and scalp cleanser. The foamed hair is rinsed with running water (2 L/min) at 40° C. for 1 minute. Excess moisture is removed using a towel and hair bundle is air-dried. After drying, a sensory evaluation is performed on the dried hair to determine its glossiness and unity. The evaluation is performed by 20 people, and the total value of the evaluation is shown. Evaluation criteria are 4: good, 3: slightly good, 2: not very good, and 1: not good.

A human hair bundle having a length of 25 cm, a width of 5.5 cm, and a weight of 10 g is thoroughly rinsed with warm water at 40° C. The hair bundle is foamed thoroughly for about 1 minute using 5 g or 10 g of the detoxifying hair and scalp cleanser composition. The foamed hair is rinsed thoroughly with running water (2 L/min) at 40° C. Excess moisture is removed using a towel and hair bundle is dried with warm air from a hair dryer. After drying, a sensory evaluation is performed on the dried hair to determine its elasticity. The evaluation is performed by 25 people, and the total value of the evaluation is shown. Evaluation criteria are 4: significant improvement in elasticity, 3: improvement of elasticity and stiffness is seen, 2: a slight improvement in elasticity, 1: no improvement of elasticity, and 0: lost elasticity.

### Example 3. Evaluation of Cleanser Efficacy Using FTIR

Bleached hair samples may be obtained. Some of the bleached hair samples may be treated with the detoxifying hair and scalp cleanser compositions described herein. For both the treated and untreated samples cross-sections will be prepared using a cryostat and the cross sections will be examined using Attenuated Total Reflection Fourier Transform Infrared Spectroscopy (ATR-FTIR). Using this technique hyperspectral images with be produced which may be used to visualize the lipid content inside the hair fiber, assess hair damage inside the hair fibers (i.e., chemical treatment, or UV exposure), identify hair structure modifications (i.e., protein conformation, or lipid organization), evaluate hair hydration (i.e., water content), visualize active penetration inside the hair fiber, and determine the efficacy of the cleanser for clearing sebum and build-up from the hair strand.

Single fibers of both the treated and untreated samples may scanned longitudinally using ATR-FTIR, which may provide infrared images of roughly the outer 1-2 microns of the fiber surface. These hyperspectral images may be used to determine the lipid content in the cuticle, investigate the hair damage along the fiber (i.e., chemical treatment, or UV exposure), analyze the deposition of an active ingredient along the hair fiber, identify hair structure modifications (i.e., protein conformation, or lipid organization), evaluate hair hydration (i.e., water content), and determine the efficacy of the cleanser for clearing sebum and products from the hair strands.

### Example 4: Evaluation of Metal Reduction/Chelation

Bleached hair samples may be obtained. Some of the bleached hair samples may be treated with the detoxifying hair and scalp cleanser compositions described herein. Both the treated and untreated samples may be rinsed with hard water and examined using Time-Of-Flight Secondary Ion Mass Spectroscopy (TOF-SIMS). Spectra may be obtained for calcium, magnesium, copper, aluminum, iron, chromium, zinc, nickel, cadmium, lead, and mercury.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A detoxifying hair and scalp cleanser, comprising:
one or more solvents;
one or more peptides;
one or more hair conditioners;
one or more skin conditioners;
one or more detoxifying agents;
one or more cleansing agents; and
one or more cosmetically or dermatologically acceptable excipients;
wherein the one or more peptides comprises a peptide sequence comprising SEQ ID NO: 75, SEQ ID NO: 400, SEQ ID NO: 401, SEQ ID NO: 402, SEQ ID NO: 409, SEQ ID NO: 412, SEQ ID NO: 432, SEQ ID NO:1131, or a combination thereof.

2. The cleanser of claim 1, wherein the concentration (% w/w) of the one or more peptides in the hair and scalp cleanser is between about 0.0001% to about 1%.

3. The cleanser of claim 1, wherein the one or more hair conditioners is selected from the group consisting of: panthenol, salicylic acid, Rubus ideaus (raspberry) leaf extract, Carthamus tinctorius (safflower) seed extract, hydroxypropylgluconamide, hydroxypropylammonium gluconate, hydrolyzed hyaluronic acid, sodium hyaluronate, or combinations thereof.

4. The cleanser of claim 1, wherein the one or more skin conditioners is selected from the group consisting of: caprylyl glycol, maltooligosyl glucoside, hydrogenated starch hydrolysate, Opuntia ficus-indica stem extract, saccharide isomerase, alpha-glucan oligosaccharide, Alteromonas ferment extract, Anetholea anisata leaf extract, guar hydroxypropyltriammonium chloride, or combinations thereof.

5. The cleanser of claim 1, wherein the one or more detoxifying agents is an absorbent.

6. The cleanser of claim 5, wherein the absorbent is a charcoal powder.

7. The cleanser of claim 1, wherein the one or more cleansing agents is a surfactant.

8. The cleanser of claim 7, wherein the surfactant is selected from the group consisting of: an anionic surfactant, an amphoteric surfactant, a non-ionic surfactant, a biosurfactant, or combinations thereof.

9. The cleanser of claim 8, wherein the surfactant comprises at least one anionic surfactant selected from the group consisting of: sodium methyl 2-sulfolaurate, sodium lauryl sulfoacetate, disodium 2-sulfolaurate, alkylbenzene sulfonates, ammonium lauryl sulfate, ammonium lauryl sulfate, ammonium xylenesulfonate, sodium C₁₄₋₁₆ olefin sulfonate, sodium cocoyl sarcosinate, sodium laureth sulfate, sodium lauryl sulfate, sodium lauryl sulfoacetate, sodium myreth sulfate, sodium xylenesulfonate, TEA-dodecylbenzenesulfonate, ethyl PEG-15 cocamine sulfate, dioctyl sodium sulfosuccinate, or combinations thereof.

10. The cleanser of claim 8, wherein the surfactant comprises at least one amphoteric surfactant selected from the group consisting of: cocamindopropyl hydroxysultaine, cocamidopropyl betaine, coco betaine, cocoamphoacetate, cocoamphodipropionate, disodium cocoamphodiacetate, disodium cocoamphodipropionate, lauroamphoacetate, sodium cocoyl isethionate, or combinations thereof.

11. The cleanser of claim 1, wherein the one more or more cosmetically or dermatologically acceptable excipients is selected from the group consisting of: a humectant; a chelating agent; a buffer or a preservative, or combinations thereof.

12. The cleanser of claim 11, wherein the humectant is selected from the group consisting of: glycerin, propylene glycol, propanediol, or isopentyldiol.

13. The hair and scalp cleanser of claim 11, wherein the chelating agent is selected from the group consisting of: sodium phytate, EDTA, sodium gluconate, trisodium dicarboxymethyl alaninate, tetrasodium glutamate diacetate, or caprylhydroxamic acid, or combinations thereof.

14. The cleanser of claim 11, wherein the buffer is selected from the group consisting of: citric acid, lactic acid, glycolic acid, malic acid, tartaric acid, L-arginine, sodium hydroxide, potassium hydroxide, or combinations thereof.

15. The cleanser of claim 11, wherein the preservative is selected from the group consisting of: phenoxyethanol, caprylhydroxamic acid, potassium sorbate, ethylhexylglycerin, gluconolactone, sodium benzoate, benzoic acid, or combinations thereof.

16. The cleanser of claim 1, wherein the concentration (% w/w) of the one or more solvents in the cleanser is between about 50% to about 90%.

17. The cleanser of claim 1, wherein the concentration (% w/w) of the one or more hair conditioners in the cleanser is between about 0.01% to about 10%.

18. The cleanser of claim 1, wherein the concentration (% w/w) of the one or more skin conditioners in the cleanser is between about 0.01% to about 5%.

19. The cleanser of claim 1, wherein the concentration (% w/w) of the one or more cleansing agents in the cleanser is between about 0.1% to about 20%.

20. The cleanser of claim 1, wherein the concentration (% w/w) of the one or more cosmetically or dermatologically acceptable excipients in the cleanser is between about 0.1% to about 30%.

21. The cleanser of claim 1, wherein the pH of the cleanser is between about 4 and about 6.

22. A method for detoxifying the hair and scalp using the cleanser of claim 1


SEQUENCE LISTING (1131 sequences)

SEQ ID NO: 1-74 - skipped (74 entries)

SEQ ID NO: 75 - protein, 10 aa, synthetic construct
CLPCLPAASC

SEQ ID NO: 76-399 - skipped (324 entries)

SEQ ID NO: 400 - protein, 13 aa, synthetic construct
GGVCGPSPPCITT

SEQ ID NO: 401 - protein, 11 aa, synthetic construct
FCGFPSCSTSC

SEQ ID NO: 402 - protein, 12 aa, synthetic construct
CTPPSCCQLHHA

SEQ ID NO: 403-408 - skipped (6 entries)

SEQ ID NO: 409 - protein, 10 aa, synthetic construct
GGVCGPSPPC

SEQ ID NO: 410-411 - skipped (2 entries)

SEQ ID NO: 412 - protein, 10 aa, synthetic construct
CGPSPPCITT

SEQ ID NO: 413-431 - skipped (19 entries)

SEQ ID NO: 432 - protein, 10 aa, synthetic construct
CEPAICEPSC

SEQ ID NO: 433-950 - skipped (518 entries)

SEQ ID NO: 951 - protein, 10 aa, synthetic construct
SCCAPVYCCK

SEQ ID NO: 952-1130 - skipped (179 entries)

SEQ ID NO: 1131 - protein, 11 aa, synthetic construct
CQCSCCKPYCS